(19) United States
(12) Patent Application Publication
(10) Pub. No.: US 2026/0289626 A1
(43) Pub. Date: Sep. 24, 2026

(54) SYSTEM

(71) Applicant: SoftBank Group Corp., Tokyo (JP)

(72) Inventor: Junya NOMURA, Tokyo (JP)

(73) Assignee: SoftBank Group Corp., Tokyo (JP)

(21) Appl. No.: 19/567,341

(22) Filed: Mar. 16, 2026

(30) Foreign Application Priority Data
Mar. 19, 2025 (JP) 2025-045168

(51) Int. Cl. G06Q 30/0601; G16H 20/60

(52) U.S. Cl. CPC G06Q 30/0623; G06Q 30/06333; G16H 20/60

(57) ABSTRACT

A system includes a processor that is configured to receive, as input from a user, budget information, disliked ingredient information, and allergen information to be avoided, analyze the received information and, based on a result of the analysis, generate a prompt sentence that instructs a generative artificial intelligence model to generate a dinner recipe, and identify ingredients required for the generated recipe and prepare an order-ready state of the ingredients through an electronic commerce platform.

### CROSS-REFERENCE TO RELATED APPLICATION

This application is based on and claims priority under 35 USC 119 from Japanese Patent Application No. 2025-045168 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional recipe recommendation systems and online grocery ordering systems generally require the user to manually search for recipes, manually adjust such recipes to accommodate personal constraints such as budget, disliked ingredients, and allergens, and then manually select and order the corresponding ingredients from an electronic commerce platform. As a result, the user is forced to expend significant time and effort on meal planning and ingredient purchasing operations. Furthermore, many existing systems do not utilize generative artificial intelligence models in a structured manner, and therefore cannot flexibly generate dinner recipes tailored to individual dietary preferences, health conditions, or emotional states of the user. In addition, known systems typically do not integrate learning of the user's dietary preferences and health condition into the recipe generation process, nor do they adapt the recipe presentation method based on the user's emotion, thereby limiting personalization and user satisfaction.

### SUMMARY

To solve the above-described problems, an aspect of the invention provides a system comprising a processor, wherein the processor is configured to receive, as input from a user, budget information, disliked ingredient information, and allergen information to be avoided. The processor is further configured to analyze the received information and, based on a result of the analysis, generate a prompt sentence that instructs a generative artificial intelligence model to generate a dinner recipe. The processor is also configured to identify ingredients required for the generated recipe and prepare an order-ready state of the ingredients through an electronic commerce platform. According to another aspect of the invention, the processor is configured to use the generative artificial intelligence model to learn dietary preferences and a health condition of the user and to optimize the recipe based on a result of the learning. According to still another aspect of the invention, the processor is configured to recognize an emotion of the user and to optimize a presentation method of the recipe based on the emotion.

The term “budget information” refers to information indicating a monetary amount or range that a user is willing or able to spend for purchasing ingredients associated with one or more recipes within a specified period or for a specified number of meals.

The term “disliked ingredient information” refers to information indicating one or more ingredients that a user does not wish to include in a recipe, regardless of allergy, for reasons such as personal taste, cultural preference, or dietary restriction.

The term “allergen information to be avoided” refers to information indicating one or more substances or ingredients that may cause an allergic reaction in a user and that are to be excluded from recipes and ingredient selections.

The term “prompt sentence” refers to a text string or structured textual instruction that is input to a generative artificial intelligence model for the purpose of causing the generative artificial intelligence model to generate one or more dinner recipes that satisfy conditions specified in the text string or structured textual instruction.

The term “generative artificial intelligence model” refers to a software-implemented model, such as a machine learning or deep learning model, that generates new content, including at least recipe text, based on an input prompt sentence and learned parameters obtained through training on example data.

The term “dinner recipe” refers to information describing at least one meal intended to be consumed as an evening or main meal, including, for example, a title, a list of ingredients with quantities, and one or more preparation or cooking steps.

The term “ingredients required for the generated recipe” refers to specific food items, seasonings, and other edible components, together with their quantities and units, that are necessary to prepare a dinner recipe generated by the generative artificial intelligence model.

The term “order-ready state” refers to a state in which a set of ingredients has been selected and registered in association with a user account on an electronic commerce platform, such that the user can place an order for the ingredients with fewer operations than manual search and selection, including, for example, a state in which the ingredients are stored in a virtual shopping cart.

The term “electronic commerce platform” refers to an online system or service, accessible via a network, that enables users to purchase goods, including ingredients, by performing electronic transactions through user accounts, virtual shopping carts, and payment mechanisms.

The term “dietary preferences” refers to tendencies or patterns relating to the types of foods, cuisines, ingredients, nutritional profiles, or preparation methods that a user tends to prefer or avoid, as inferred or learned from user input, historical behavior, or feedback.

The term “health condition of the user” refers to information related to a physical or physiological state of a user, including, for example, body weight, medical conditions, dietary restrictions, activity level, or health goals such as weight loss or muscle gain.

The term “optimize the recipe” refers to adjusting, selecting, or re-generating one or more recipes so as to better satisfy one or more criteria associated with the user, including at least dietary preferences, health condition, budget information, and ingredient-related constraints.

The term “emotion of the user” refers to an affective state of the user, such as happiness, sadness, stress, or fatigue, which may be recognized or inferred from user input, sensor data, behavioral patterns, or external interfaces.

The term “presentation method of the recipe” refers to a manner in which a recipe is output or displayed to a user, including, for example, layout, level of detail, selection of images, textual tone, ordering of information, and timing or modality of delivery.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

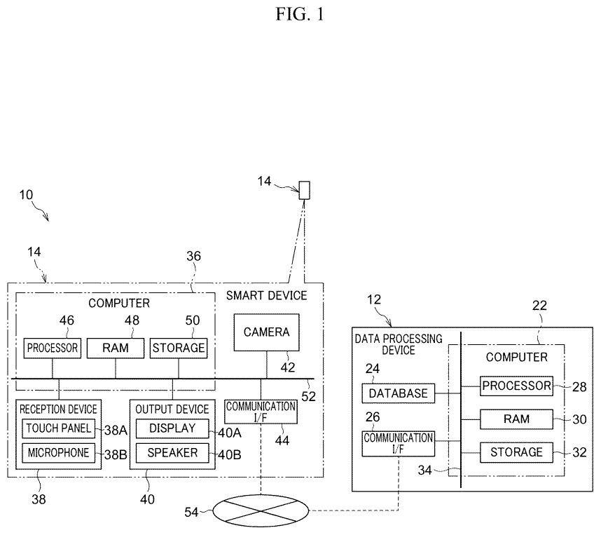

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

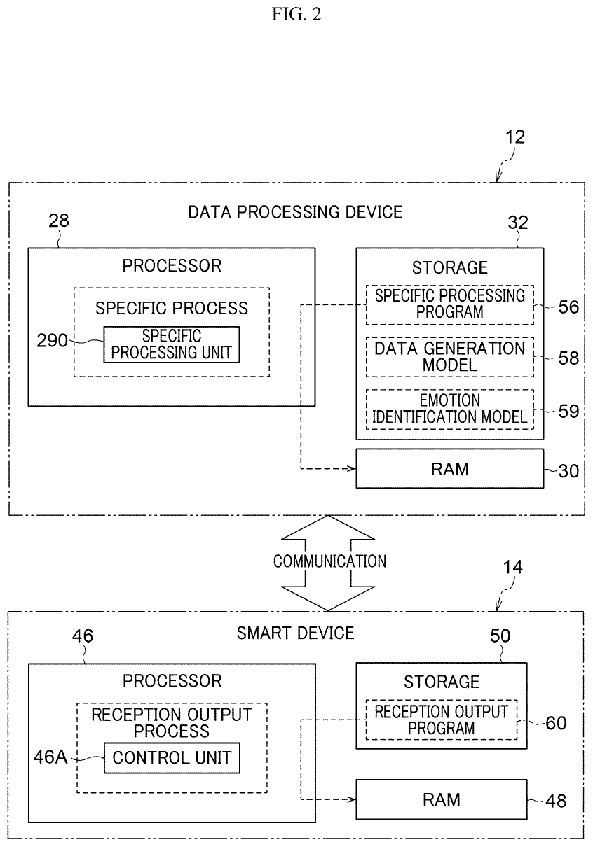

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented meal planning systems typically rely on fixed rule sets, static recipe databases, or simple filtering logic executed by application software. Such systems suffer from several technical limitations when executed on general-purpose computing hardware. First, when a large volume of recipe data and user constraint data (including budget, preferences, and allergen restrictions) is processed, the server often executes multiple redundant database queries and rule evaluations, which increases processor load and memory consumption, causes unnecessary network traffic to external commerce services, and leads to latency in user-facing responses. Second, existing systems generally do not integrate a generative AI model in a technically structured manner; instead, they may invoke a model with loosely defined or ad hoc prompts, resulting in unstructured outputs that require extensive post-processing, additional parsing steps, and repeated error correction, thereby degrading overall computational efficiency.

Moreover, in many known architectures, the transformation from high-level user constraints into concrete, machine-usable purchase information is fragmented across multiple modules: one component may compute a meal plan, another component may separately compute ingredient quantities, and yet another may map those ingredients to purchasable items. This fragmentation often leads to duplicated data transformations, inconsistent internal representations of ingredients and quantities, and increased risk of mismatches between the generated plan and the items added to an online shopping cart. As a result, the server must perform additional corrective processing, re-query external services, or request repeated user input, placing further demands on processing resources and network bandwidth.

In addition, traditional systems typically do not treat the interaction with a generative AI model as an adaptive, learnable process tightly integrated into the server's control flow and data structures. User-specific patterns such as historical selections, long-term dietary preferences, and health-related constraints are often processed outside the model or not fed back to the model in a systematic way. Consequently, the system cannot effectively reduce repeated computation for similar users or similar constraints, and cannot leverage model-side adaptation to improve the quality and structure of future outputs. This leads to inefficient use of computing resources and sustained latency across multiple sessions.

Furthermore, while some systems attempt to react to user state, such as stress or dissatisfaction, they typically adjust only user interface decoration and do not alter the underlying data generation or selection pipeline. They do not integrate emotional state signals into the prompt design or output control of the generative AI model, and therefore cannot optimize the internal generation and selection steps executed by the processor and memory subsystems. This again forces the system to rely on post-hoc presentation changes, which do not reduce the computational cost of initial plan generation and may still require repeated generation cycles.

Accordingly, there is a need for a computer-implemented technique that improves how a server processor orchestrates (i) the acquisition and structuring of user constraints, (ii) the generation of prompt sentences for a generative AI model, (iii) the structured generation and validation of meal plan information, (iv) the aggregation and normalization of ingredient data, and (v) the mapping to and interaction with external product sales services, in order to reduce redundant computation, improve data consistency across internal modules, and decrease the number of network transactions, while enabling adaptive behavior based on user history and user emotional state.

The specific processing by the specific processing unit 290 of the data processing device 12 in Example 1 is realized by the following means.

The present invention provides a server comprising a processor and a memory storing instructions that, when executed by the processor, cause the processor to perform operations including: receiving, via an input interface, budget information, preference information, and allergen information from a user; generating a structured condition including the budget information, the preference information, and the allergen information; generating, based on the structured condition, a search request for searching meal information stored in an information storage device; acquiring candidate meal information by executing the search request; generating, in a deterministic and machine-interpretable format, a prompt sentence including the candidate meal information and the structured condition; inputting the prompt sentence to a generative AI model so as to cause the generative AI model to generate meal plan information for a predetermined period; extracting ingredient information for each meal included in the meal plan information; aggregating quantity information for each type of ingredient based on the ingredient information to generate purchase candidate ingredient information in a normalized internal representation; transmitting, based on the purchase candidate ingredient information, a product search request to an external product sales service through a communication network; acquiring product information from the external product sales service; selecting, in accordance with the normalized internal representation, purchase target product information for each ingredient from the product information; adding the purchase target product information to purchase management information of the external product sales service so as to generate purchase preparation information representing a state immediately before ordering; and generating output information for presentation to a user device, the output information including the meal plan information and the purchase preparation information. This enables the server to reduce redundant processing and network transactions by maintaining a unified, structured data flow from user constraints to meal plan information and purchase preparation information, to invoke the generative AI model using prompt sentences that are tightly coupled to database search results and internal representations, to minimize post-processing and error correction of model outputs, to improve consistency between generated meal plans and purchasable items, and to enhance overall computational efficiency and responsiveness of the computer system executing the meal planning and purchasing operations.

The term “budget information” refers to information indicating an upper limit or range of expenditure that a user allows for purchasing materials or products associated with meal planning over a predetermined period.

The term “preference information” refers to information indicating a user's likes and dislikes regarding meals, ingredients, cooking styles, or other culinary attributes, including but not limited to preferred ingredients, disfavored ingredients, and dietary patterns.

The term “allergen information” refers to information indicating one or more substances, ingredient types, or categories that may cause an allergic reaction for the user and that are to be avoided in meal planning and material selection.

The term “input interface” refers to a hardware and software combination configured to receive data from a user or a user device, including graphical user interfaces, application programming interfaces, communication ports, and associated input processing logic.

The term “structured condition” refers to a machine-readable data set derived from user-provided information, the data set including at least budget information, preference information, and allergen information organized in a normalized format suitable for database querying and prompt generation.

The term “information storage device” refers to a physical or virtual storage resource, such as a memory device or a storage subsystem, configured to store digital data including meal information, recipe information, ingredient information, and related metadata.

The term “meal information” refers to information representing one or more meals, including at least a meal name, ingredient data, preparation instructions, cost estimates, and optionally nutritional attributes.

The term “search request” refers to a machine-implemented request generated by the processor to access and retrieve data from an information storage device or external system, the request typically including one or more search conditions, filter conditions, or query parameters.

The term “candidate meal information” refers to meal information retrieved from an information storage device in response to a search request and preliminarily determined as suitable for use in generating a meal plan based on a structured condition.

The term “prompt sentence” refers to a text or structured text sequence generated by the processor and configured to be provided as input to a generative AI model, the text including at least one condition, one or more constraints, and optionally candidate meal information to guide the generative AI model's output.

The term “generative AI model” refers to a machine learning based information processing model, such as a neural network language model, configured to generate new data including text or structured information in response to an input prompt sentence.

The term “meal plan information” refers to information representing a plan of multiple meals over a predetermined period, the plan including at least identification of meals, scheduling of meals, and associated ingredient and cost information.

The term “ingredient information” refers to information specifying one or more components used in preparing a meal, including ingredient names, categories, quantities, and optionally units of measure and nutritional attributes.

The term “quantity information” refers to information indicating an amount of an ingredient, expressed in units such as weight, volume, or count, and suitable for aggregation and normalization across multiple meals.

The term “purchase candidate ingredient information” refers to ingredient-related data that has been aggregated and normalized across a meal plan and that represents preliminary target items to be fulfilled by purchasable products from an external product sales service.

The term “normalized internal representation” refers to a data format for ingredients and quantities unified across different meals and data sources, wherein ingredient names, units, and identifiers are standardized to enable consistent aggregation, comparison, and mapping to product information.

The term “communication network” refers to a wired or wireless data transfer infrastructure, including local area networks, wide area networks, and public networks, that enables electronic communication between the server and external systems, including external product sales services.

The term “external product sales service” refers to a remote computer-implemented commerce system that provides product information, product search functionality, and purchase management functionality, and that is accessible through a communication network.

The term “product search request” refers to a message transmitted from the server to an external product sales service, the message including one or more parameters derived from purchase candidate ingredient information to obtain corresponding product information.

The term “product information” refers to information provided by an external product sales service, including but not limited to product identifiers, product names, package sizes, prices, availability, and related metadata.

The term “purchase target product information” refers to a subset of product information selected by the processor as suitable to fulfill requirements indicated by purchase candidate ingredient information, including selected product identifiers and quantities to be purchased.

The term “purchase management information” refers to data managed by an external product sales service for handling purchase-related operations, including shopping cart contents, order identifiers, user account identifiers, and payment-related states.

The term “purchase preparation information” refers to information representing a transaction state managed by an external product sales service in which purchase target product information has been added to purchase management information and is ready for user confirmation or ordering.

The term “output information” refers to digital data generated by the processor for presentation to a user device, including at least meal plan information and purchase preparation information, formatted for display, interaction, or further processing.

The term “user device” refers to an information processing terminal, such as a personal computer, a smartphone, a tablet, or another client apparatus, operated by a user to input information to the server and to receive and present output information.

The term “historical meal information” refers to previously recorded data concerning meals associated with a user, including past meal plans, selected recipes, and consumed meals, stored for use in subsequent processing or learning.

The term “historical preference information” refers to data representing prior user choices, likes, dislikes, and modification patterns relating to meals or ingredients, accumulated over time and used to infer user-specific tendencies.

The term “feature information” refers to abstracted data or parameter values derived from historical meal information and historical preference information that characterize a user's meal preference patterns and health-related tendencies.

The term “internal parameter” refers to a trainable or configurable value within a generative AI model that influences the model's processing behavior and output, such as a weight, bias, or similar model-specific parameter.

The term “operation condition” refers to a configuration value or control parameter of a generative AI model or server process, including but not limited to temperature, sampling strategy, or context window settings that affect how outputs are generated.

The term “operation information” refers to information derived from user interactions with a user device or an application, including input sequences, selection operations, interaction timings, and navigation patterns.

The term “sensor information” refers to information obtained from one or more sensors associated with a user device or a peripheral device, including but not limited to biometric data, motion data, facial expression data, or voice data.

The term “emotional state” refers to an estimated mental or affective condition of a user, such as stress, satisfaction, or fatigue, inferred from operation information, sensor information, or a combination thereof.

The term “presentation format” refers to a structure or layout by which meal plan information or related content is displayed or conveyed to the user, including arrangement, grouping, visual emphasis, and accompanying textual explanations.

The term “display order” refers to the sequence in which elements of output information, such as meals, recipes, or recommendations, are presented to the user on a display or other output interface.

The term “emphasis degree” refers to a level or manner of visual or auditory highlighting applied to certain elements of output information, such as font size, color, position, animation, or sound characteristics.

The term “explanation content” refers to descriptive text or other explanatory data associated with meal plan information or recipes, used to clarify why particular items were selected or how they satisfy user conditions.

The term “presentation method of a recipe” refers to a combined configuration of display order, emphasis degree, and explanation content, or other presentation parameters, applied to recipe-related information for the purpose of improving user comprehension, engagement, or satisfaction.

In the following embodiments, a server, a terminal, and a user cooperate to implement the claimed system. A processor in the server executes instructions stored in a memory to realize the described functions. The server can be implemented on general-purpose computing hardware, such as a rack-mounted computer including a central processing unit (CPU), a graphics processing unit (GPU) for machine learning computation, a main memory (DRAM), a non-volatile storage device (solid state drive), and a network interface controller connected to a communication network. The terminal can be a personal computer, a smartphone, or a tablet including a display, an input device, a wireless or wired network interface, and an operating system. The server can execute server-side software such as an operating system, a web server, and an application framework, for example a web framework implemented in a high-level programming language. The terminal can execute a web browser or a native application.

The server stores meal information, ingredient information, product information mappings, user history information, and learned model parameters in one or more information storage devices. The server can use a relational database management system, such as a generic SQL database, to store structured data in tables. The server can use an object storage or a file system to store unstructured data, including model configuration files, tokenizer vocabularies, and log information. The server can use database drivers or object-relational mapping software to access the database. By using these components, the server maintains consistent, indexed, and query-efficient data structures that reduce lookup time and redundant data transfers.

The server uses a generative AI model. In one embodiment, the generative AI model is a neural network language model having a transformer architecture characterized by a plurality of stacked layers, each layer including a multi-head self-attention mechanism and a feed-forward network. The server stores model parameters, such as weight matrices and bias vectors, in a model parameter storage area in the memory or in a dedicated accelerator memory. The server uses a tokenizer to convert a prompt sentence into a sequence of tokens, each token mapped to an integer identifier. The server then passes the token sequence to the transformer model, which performs matrix multiplications, non-linear activation functions, and attention weight computations to produce output token sequences representing generated text. The server can use an inference engine library optimized for vector operations and GPU execution, thus improving the processing speed and reducing latency.

The server uses an explicit, structured data flow to connect user constraints, database searches, prompt sentence construction, generative AI model invocation, and external product service integration. The server maintains internal data structures representing a structured condition derived from budget information, preference information, and allergen information. For example, the server maintains a condition object including fields for total budget, prohibited ingredient categories, allergen tags to exclude, desired period length, and preferred cuisine types. By normalizing and structuring user input into such an internal representation, the server reduces ambiguity, enables deterministic query generation, and decreases the number of corrective passes needed when interacting with the generative AI model.

The server performs data processing operations on this structured condition using conventional algorithms executed on the CPU. The server maps user-entered ingredient names to canonical ingredient identifiers using a controlled vocabulary table and string normalization algorithms. The server maps allergen information to standardized allergen categories stored in an allergen table. The server computes a per-day or per-meal budget by dividing the total budget by the period length. The server stores the resulting structured condition in the relational database, thereby allowing reuse of the structured condition in subsequent processing or future user sessions. This persistence reduces the need to reconstruct conditions from raw input and contributes to processing efficiency.

The server searches an internal meal information database using the structured condition. The server constructs a search request in the form of an SQL query or an equivalent query representation that references indexed columns, such as ingredient identifiers, allergen flags, and estimated cost fields. The server uses join operations and filter predicates to select candidate meal information entries that satisfy the structured condition. The server can also use precomputed cost-per-portion values stored in the database to quickly filter meals that exceed the per-meal budget. This approach reduces CPU load and database scan time compared to computing costs on the fly for all recipes.

The server generates a prompt sentence for the generative AI model using the structured condition and the candidate meal information. The server assembles a multi-part prompt sentence containing: (i) an explicit description of user constraints, (ii) a short summary of available candidate meals, and (iii) a strict output format description. The server includes explicit instructions that the generated text should be structured by day and meal type and should not include ingredients associated with disfavored items or allergens. For example, in one embodiment, the server generates a prompt sentence as follows:

“User conditions:
Total budget: 5000 yen for 7 days
Disliked ingredients: green peppers
Allergens to avoid: nuts

Candidate recipes:

[Here, list candidate dishes such as simple home-style dishes, with their main ingredients and approximate costs.]

Task:

Generate a 1-week meal plan (7 days) with breakfast, lunch, and dinner each day. Exclude any dishes that contain green peppers or nuts. Keep the total estimated cost within 5000 yen. Use as many candidate recipes as possible, but you may create new simple recipes if necessary. For each meal, provide: the name of the dish, a list of ingredients, the quantity of each ingredient per serving, and an estimated cost. Present your answer in a clear, structured format.”

By constraining the prompt sentence to be deterministic, explicit, and aligned with internal data structures, the server improves the reliability and structure of the generative model's output. This reduces the need for repeated calls to the generative AI model and decreases parsing complexity.

The server uses a particular neural network configuration and training paradigm to improve adaptation to individual users. The server fine-tunes or conditions the generative AI model on user-specific feature information derived from historical meal information and historical preference information. The server constructs learning prompt sentences that embed sequences of past user selections, ratings, and modifications, along with inferred health-related preferences. The server uses a loss function, such as a cross-entropy loss, computed between generated sequences and target sequences representing user-accepted plans. During training, the server updates internal parameters of the neural network using gradient-based optimization methods, such as stochastic gradient descent or its variants, modifying weights in attention layers and feed-forward layers. The server may apply data augmentation techniques, such as random substitution of similar ingredients or slight variations of budgets, to increase the robustness of learned patterns. As a result, the generative AI model learns to internally encode feature information that better reflects individual user tendencies, thereby producing more accurate and relevant meal plans with fewer corrective iterations.

The server aggregates ingredient information across the generated meal plan information. The server uses an ingredient-normalization module that maps different textual forms of an ingredient to a canonical identifier. The server performs unit conversion using a conversion table to convert volume units and count units to standard base units (for example, grams) whenever possible. The server uses associative arrays or hash maps to accumulate quantities for each canonical ingredient identifier across all meals. By using canonical identifiers and base units, the server prevents double-counting and inconsistent unit aggregation, thus improving the accuracy of ingredient totals.

The server maps aggregated ingredient quantities to purchasable products on an external product sales service. The server maintains a mapping table between canonical ingredient identifiers and product category identifiers used by the external service. The server constructs a product search request using category identifiers and desired package sizes. The server may use a simple optimization algorithm, such as integer rounding based on package size multiples, to determine how many packages to purchase per ingredient. The server can compute total expected cost by summing the product of unit price and quantity for each selected item. By structuring this mapping as a deterministic algorithm using normalized data structures, the server reduces the number of failed matches and the number of repeated product searches.

The server interacts with external product sales services using standardized application programming interfaces. The server can send HTTP requests to endpoints providing search and shopping cart operations. The server transmits product identifiers and requested quantities, and the external service updates purchase management information, such as a shopping cart. The server receives responses containing product information and cart identifiers and stores this data in the relational database. By caching selected product mappings and cart states, the server decreases network usage for repeated shopping sessions with similar ingredient sets.

The server presents meal plan information and purchase preparation information to the user via the terminal. The terminal displays structured outputs received from the server using a graphical user interface. The terminal can display a list or calendar view of the meal plan, and an indication that all necessary materials have been prepared in a shopping cart on the external product sales service. When the user modifies preferences or requests a new plan, the terminal transmits updated information to the server, which updates the structured condition and repeats the processing described above.

The server adapts processing and presentation based on an estimated emotional state of the user. The terminal can collect operation information such as scrolling speed, hesitation time, or frequency of plan modifications, and sensor information such as facial expression patterns captured by a camera or voice characteristics captured by a microphone. The server uses a separate classifier model, such as a smaller neural network, that processes extracted features (for example, average interaction latency, facial expression embeddings, or voice pitch statistics) and outputs an emotional state category. The server then adjusts the next prompt sentence to the generative AI model by including constraints and meta-instructions, such as:

“The user appears tired and stressed. Please generate simpler, low-effort recipes with fewer preparation steps, and provide reassuring explanations about the ease of cooking each dish.”

By including emotional-state-based constraints in the prompt sentence, the server controls not only how the result is displayed but also how the generative model structures the underlying meal plan. This reduces the need for multiple generations and decreases the cognitive load on the user by adapting both computation and output content.

The server improves computer technology in multiple ways. By using structured conditions and canonical ingredient identifiers, the server reduces the dimensionality and variability of inputs to the generative AI model and to external services, thereby improving computational efficiency. By designing prompt sentences that closely reflect internal data structures and by validating generated output against those structures, the server minimizes inconsistent or malformed outputs, which reduces the number of model calls and post-processing steps. By learning user-specific feature information within the generative AI model, the server lowers the number of candidate plans required before a satisfactory plan is obtained, thus decreasing CPU, GPU, and network usage for repeated sessions. By caching product mappings and by mapping ingredients to purchasable products using normalized identifiers and package-based optimization, the server reduces redundant network accesses to external product sales services and lowers overall communication load.

The server achieves technical effects such as faster response times, reduced error rates in ingredient mapping and allergen handling, and improved resource usage patterns on the computing hardware. The combination of transformer-based generative modeling, structured prompt sentence generation, and deterministic ingredient normalization algorithms yields more precise and reliable outputs than simple rule-based filtering of static recipe databases. Because the server uses internal feature representations learned by the generative AI model through fine-tuning and controlled conditioning, the server handles complex combinations of constraints that would be difficult to manage with traditional rule systems. This processing is not a mere automation of a human task but an improvement in how the computing system as a whole stores, transforms, and utilizes data.

In alternative embodiments, the server can employ different neural network architectures, such as recurrent neural networks or convolutional sequence models, provided that the model receives a prompt sentence and generates meal plan information. The server can implement the ingredient normalization module using different similarity metrics, such as edit distance or embedding-based similarity. The server can approximate unit conversion using lookup tables or parametric models. The server can adjust the period of the meal plan, the granularity of meals (for example, including snacks), or the level of nutritional detail. The server can operate without emotional-state estimation in some embodiments, or can use only operation information without sensor information.

In another embodiment, the server executes on a distributed computing platform, where a front-end server handles user input and output, and a back-end server handles database access and generative AI model inference. The servers communicate over a data center network. The generative AI model may be deployed on specialized accelerator hardware, such as dedicated inference servers. This division allows horizontal scaling of specific components, such as model inference, without changing the fundamental structure of prompt sentence generation, condition normalization, and product mapping. In yet another embodiment, the terminal executes some local pre-processing, such as basic input validation or temporary caching of meal plan fragments, to reduce server load while leaving core processing, including generative AI model invocation and product mapping, on the server.

Through these embodiments and variations, the server, terminal, and user cooperate to realize a technically specific workflow that uses a generative AI model and structured prompt sentences not merely to automate a business process but to enhance the internal operation of the computing system, including data representation, algorithmic efficiency, and network interaction patterns.

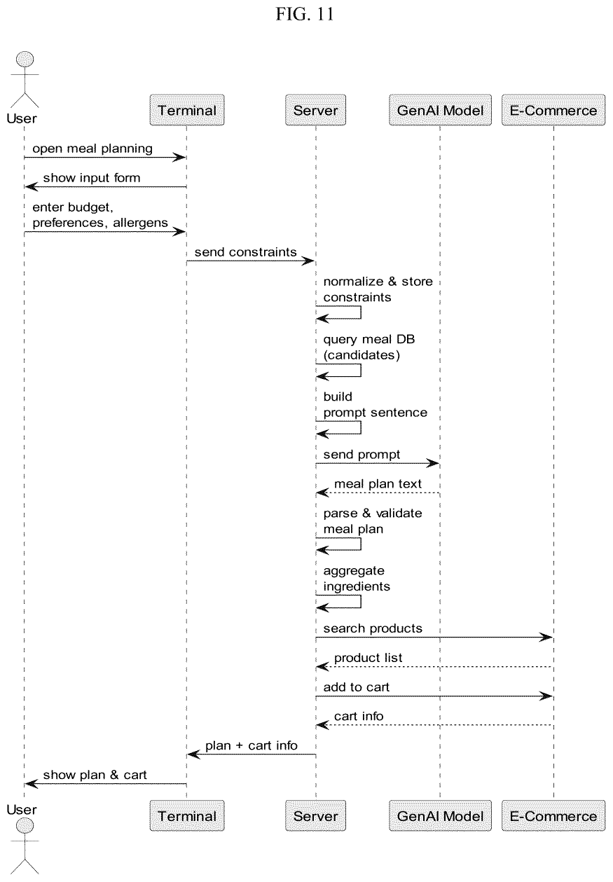

The following describes the processing flow using FIG. 11.

### Step 1

User operates the terminal to open an application screen for meal planning.

Terminal displays input fields for budget, disliked ingredients, and allergens.

Input: No prior user-specific data; only default UI configuration.

Output: A rendered input form on the terminal display.

### Step 2

User inputs concrete values such as a total budget, a list of disliked ingredients, and a list of allergens to avoid, and then confirms the input.

Terminal performs basic validation (for example, checking that the budget is numeric and that text fields are not empty) using local logic.

Input: User keystrokes and touch operations.

Output: A structured in-memory representation of the user's input, such as a key-value map.

### Step 3

Terminal converts the validated user input into a machine-readable message, for example a JSON object containing fields for budget, preference information, and allergen information.

Terminal sends this message to the server over a communication network using a request to a predefined application programming interface endpoint.

Input: In-memory map of budget and preferences.

Output: A network request containing structured user constraint data transmitted to the server.

### Step 4

Server receives the network request via a network interface and decodes the message payload using an application framework.

Server parses the structured data into internal data structures, such as objects or records, and performs server-side validation (for example, range checks on budget and normalization of ingredient text to lower case).

Input: Structured user constraint data from the terminal.

Output: A normalized condition object in server memory representing the user's budget, disliked ingredients, and allergens.

### Step 5

Server maps text strings for ingredients and allergens to canonical identifiers using lookup tables stored in a relational database.

Server executes database queries to retrieve canonical ingredient identifiers and allergen category identifiers, then replaces raw text values with these identifiers in the condition object.

Input: Normalized condition object with raw ingredient and allergen strings.

Output: An updated condition object with canonical ingredient IDs and allergen category IDs.

### Step 6

Server stores the condition object in persistent storage by executing insert or update operations on one or more database tables associated with user constraints.

Server commits the database transaction to ensure durability of the stored condition.

Input: Condition object with canonical identifiers.

Output: Database records representing the user's current constraints and an in-memory reference to these records.

### Step 7

Server constructs a search request for meal information by translating the condition object into a database query, including filter predicates on cost, ingredient IDs, and allergen flags.

Server sends this query to the meal information database through a database driver.

Input: Condition object with budget and canonical ingredient and allergen identifiers.

Output: A structured database query expressing selection conditions for candidate meals.

### Step 8

Server receives candidate meal information from the database as a result set containing fields such as meal identifiers, names, ingredient lists, cost estimates, and allergen flags.

Server converts each row of the result set into an internal meal object and collects these objects into a list of candidate meals.

Input: Database result set for the search request.

Output: A list of candidate meal objects in server memory.

### Step 9

Server evaluates whether the number and diversity of candidate meals are sufficient to cover the planned period by counting meal objects and checking category coverage.

Server sets a flag or attribute in the condition object indicating whether additional meal generation by a generative AI model is required.

Input: List of candidate meal objects and condition object.

Output: An annotated condition object indicating sufficiency of candidates and the same or updated list of candidate meals.

### Step 10

Server generates a prompt sentence for the generative AI model by combining user conditions, candidate meal summaries, and explicit output format instructions into a single textual string.

Server ensures that the prompt sentence includes constraints on total budget, forbidden ingredients, forbidden allergens, and required structure of the resulting meal plan.

Input: Condition object and list of candidate meals.

Output: A fully constructed prompt sentence in natural language.

### Step 11

Server tokenizes the prompt sentence using a tokenizer associated with the generative AI model, converting the text into a sequence of tokens represented as numeric identifiers.

Server prepares a request body for the generative AI model API, embedding the tokenized or raw prompt sentence and specifying model parameters such as maximum output length and sampling temperature.

Input: Prompt sentence text.

Output: A model inference request containing the prompt sentence and configuration parameters.

### Step 12

Server sends the model inference request to the generative AI model over a network connection or through a local inference interface.

Generative AI model processes the prompt sentence by performing neural network computations layer by layer, including self-attention, feed-forward transformations, and softmax-based token probability estimation, to generate a sequence of output tokens.

Input: Inference request with prompt sentence tokens and model parameters.

Output: A sequence of generated tokens representing the meal plan information.

### Step 13

Server receives the generated token sequence from the generative AI model and detokenizes it to reconstruct the output text.

Server parses the output text according to the instructed structure, extracting day indices, meal types, recipe names, ingredient names, quantities, and estimated costs into structured objects.

Input: Sequence of generated tokens or generated text.

Output: A structured meal plan object containing meals, ingredients, and cost information for the predetermined period.

### Step 14

Server validates the structured meal plan by checking all ingredients against the canonical ingredient table and allergen table in the database.

Server executes checks to ensure that no ingredient matches a disliked ingredient identifier and that no ingredient is associated with any forbidden allergen identifier; if violations are found, server either flags the plan or triggers a corrective process.

Input: Structured meal plan object and database tables for ingredients and allergens.

Output: A validated or flagged meal plan object, including validation status and any detected violations.

### Step 15

Server aggregates ingredient requirements across all meals in the validated meal plan by iterating over each meal and each ingredient, converting quantities to standard units, and summing quantities per canonical ingredient identifier.

Server stores the aggregated quantities in a data structure, such as a mapping from ingredient ID to total required quantity, and normalizes units using a unit conversion table.

Input: Validated meal plan object with per-meal ingredient quantities.

Output: An aggregated ingredient requirement map keyed by canonical ingredient identifiers with standardized quantities.

### Step 16

Server generates purchase candidate ingredient information by associating each ingredient identifier in the aggregated map with additional metadata such as preferred product category, default package size, and acceptable substitutes from configuration tables.

Server saves the purchase candidate ingredient information in the database for later use and reference.

Input: Aggregated ingredient requirement map and ingredient configuration tables.

Output: A list or table of purchase candidate ingredient records, each with an ingredient ID, total quantity, and associated metadata.

### Step 17

Server constructs a product search request for each purchase candidate ingredient by forming query parameters that include ingredient category identifiers, required quantities, and other constraints such as allergen-free or budget limits.

Server sends these product search requests to an external product sales service via a communication network using defined API endpoints.

Input: Purchase candidate ingredient records.

Output: One or more outgoing product search requests transmitted to the external product sales service.

### Step 18

Server receives product information responses from the external product sales service, each response containing product identifiers, prices, package sizes, and availability flags.

Server stores the product information temporarily in memory and optionally persists useful mappings to the database for caching.

Input: Product search responses from the external service.

Output: In-memory lists of product candidates associated with each ingredient.

### Step 19

Server selects purchase target product information for each ingredient by applying selection rules that consider total required quantity, package size, unit price, and allergen safety.

Server computes how many packages of each selected product are needed by dividing the total required quantity by package size and rounding up to the nearest integer, then calculates the resulting total cost.

Input: Product candidate lists and purchase candidate ingredient records.

Output: A set of purchase target product entries, each including a product identifier, number of packages, and total cost contribution.

### Step 20

Server generates purchase preparation information by assembling the selected products into a structure compatible with the external product sales service's purchase management system, such as a shopping cart representation.

Server sends a cart update request to the external product sales service API, adding the selected products with appropriate quantities to the user's shopping cart or equivalent structure.

Input: Purchase target product entries.

Output: A purchase preparation record including a cart identifier or confirmation data from the external service.

### Step 21

Server calculates summary statistics for the entire meal plan and purchase set, such as total cost, cost per day, and total number of distinct ingredients.

Server updates internal records to link the meal plan, aggregated ingredients, and purchase preparation information, enabling consistent retrieval and display.

Input: Structured meal plan object and purchase preparation record.

Output: A unified plan summary object containing both dietary and purchasing overview data.

### Step 22

Server generates output information for the terminal by formatting the meal plan, ingredient summaries, and purchase preparation information into a response payload that can be directly rendered by the terminal's user interface.

Server includes human-readable explanations when necessary, such as reasons for excluding certain ingredients or how the total cost meets the budget constraint.

Input: Unified plan summary object.

Output: A structured response message containing display-ready meal plan and shopping cart information.

### Step 23

Terminal receives the response message from the server and parses the structured data into internal UI components.

Terminal renders the weekly meal plan, including daily meals, ingredients, and estimated costs, and displays a control element allowing the user to navigate to the external product sales service or review the prepared cart contents.

Input: Structured response message from the server.

Output: A rendered user interface showing the meal plan and purchase preparation status.

### Step 24

User reviews the displayed meal plan and purchase information on the terminal, and may optionally adjust preferences or request a modified plan by interacting with controls.

Terminal captures these new user interactions as updated constraints or modification commands and prepares them for transmission as a new or updated request.

Input: Visual output on the terminal and user actions.

Output: Modified constraint data or commands that can initiate a new iteration of the processing flow on the server.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented meal planning and ingredient ordering systems mainly rely on fixed rule sets, manually curated recipe databases, and static mapping from user inputs to pre-defined meal plans. In such systems, a processor typically receives basic user inputs, selects recipes from a limited database, and generates a simple shopping list. However, these approaches suffer from several technical drawbacks when implemented at scale in modern distributed computing environments.

First, conventional systems do not efficiently utilize generative AI models in a way that is tightly integrated with downstream data processing pipelines and external commerce services. In many cases, generative models are invoked in an ad hoc fashion, and the textual output is manually or semi-manually interpreted, resulting in high latency, inconsistent data structures, and significant processor and memory overhead to normalize and reconcile the generated content with ordering systems.

Second, existing systems lack an integrated mechanism at the processor level to transform unstructured natural language outputs from generative AI models into normalized, aggregated ingredient data that is directly consumable by electronic commerce platforms. As a result, separate, often redundant processing components must be implemented to perform name normalization, unit conversion, and quantity aggregation, which leads to increased computational complexity, inefficient use of memory and storage resources, and error-prone mappings between recipe data and product catalogs.

Third, traditional systems do not provide an optimized feedback loop between user interactions and the generative AI model in a way that adaptively refines prompt sentences and recipe outputs. Learning from user meal histories, preferences, and health conditions is often done as a separate analytics layer, if at all, and is not systematically tied to the generation of prompt sentences and recipe content. Consequently, the processor is unable to use prior user data effectively to guide the generative AI model, leading to suboptimal personalization and redundant computation for each new request.

Fourth, user emotion and context are typically ignored in existing recipe presentation interfaces. Even when some form of emotion recognition is present, it is implemented as a separate front-end feature and is not integrated into the core processing logic that controls the ordering, expression, and substance of recipe presentation. This separation results in fragmented state management, duplicated rendering logic, and inefficient communication between client and server components.

Therefore, there is a need for an improved computer-implemented system and processing method in which a processor: (i) generates and supplies structured prompt sentences to a generative AI model based on normalized user condition information; (ii) automatically converts the model's generated recipes into normalized, aggregated ingredient-total information; (iii) programmatically associates the ingredient-total information with product information from an external commerce service and prepares order data up to an order-ready state; and (iv) adaptively optimizes both the prompt sentences and the presentation of recipes based on accumulated user data and estimated emotional state. Such a system would improve the efficiency, reliability, and scalability of server-side processing for meal planning and ingredient ordering, and reduce the computational and integration overhead typically associated with generative AI based solutions.

The specific processing by the specific processing unit 290 of the data processing device 12 in Application Example 1 is realized by the following means.

The present invention provides a server comprising a processor configured to receive, from a communication terminal operated by a user, condition information including numerical information regarding a budget and attribute information regarding user preferences and restrictions; analyze the condition information and, based on a result of the analysis, generate a prompt sentence that instructs generation of candidate dinner menus for a plurality of days and ingredient groups corresponding to the candidate dinner menus; supply the prompt sentence as input to a generative artificial intelligence model and execute inference processing by the generative artificial intelligence model to obtain dinner menus for the plurality of days that satisfy the condition information and ingredient information included in the dinner menus; aggregate the ingredient information included in the dinner menus for the plurality of days and generate, by performing data processing including normalization of ingredient names, conversion of units, and summation of quantities, ingredient-total information indicating total quantities per ingredient over the plurality of days; generate, based on the ingredient-total information, order data including product identification information and order quantities corresponding to the ingredients by associating the ingredients with product information provided from an external commerce service, and transmit the order data to an application programming interface of the commerce service so as to prepare an order to a state immediately before placement; present the dinner menus for the plurality of days and the order data to the user via the communication terminal, receive confirmation and correction operations from the user, and reflect a result of the correction operations in the ingredient-total information and the order data; use the generative artificial intelligence model or another learning model to accumulate information including user meal history information, preference information, and health condition information, and, based on learning processing performed on the accumulated information, optimize the prompt sentence and contents of the dinner menus for the plurality of days for each user; and estimate an emotional state of the user based on input operation information, voice information, or image information acquired from the communication terminal and, in accordance with the estimated emotional state, change at least one of a presentation order, a presentation expression, and proposal contents of the dinner menus for the plurality of days so as to dynamically control a presentation method of recipes. This enables improvement of computer functionality by providing an integrated server-side mechanism that efficiently orchestrates generation, structuring, aggregation, and commerce-oriented transformation of recipe data produced by a generative artificial intelligence model, while adaptively optimizing prompts and user interface behavior based on learned user characteristics and emotional state, thereby reducing processing redundancy, lowering latency, and enhancing reliability and scalability of meal planning and ordering operations in a networked environment.

The term “processor” refers to a hardware logic element or a combination of hardware and software elements configured to execute instructions for performing data processing, control, and communication functions in the system.

The term “system” refers to a collection of one or more processors, memories, communication interfaces, and associated software modules that cooperate to implement the meal planning, recipe generation, ingredient aggregation, and order preparation functions described herein.

The term “user” refers to a human operator who interacts with the system to provide input conditions, review generated menus, and confirm or modify ingredient ordering.

The term “communication terminal” refers to an electronic device, such as a mobile device, a tablet device, or a personal computing device, configured to communicate with the server over a communication network and to present user interfaces for input and output operations.

The term “condition information” refers to digital data received from the user that specifies constraints or preferences for meal planning, including at least numerical information regarding a budget and attribute information regarding user preferences and restrictions.

The term “budget” refers to a numerical constraint indicating an upper limit or target range for an expenditure amount associated with purchasing ingredients required for the generated dinner menus.

The term “user preferences” refers to attributes indicating the user's likes, dislikes, dietary habits, or desired characteristics of meals, including at least disliked ingredients.

The term “restrictions” refers to constraints on ingredient selection or meal composition, including at least allergen-related restrictions and dietary limitations imposed by health conditions or personal choices.

The term “disliked ingredients” refers to ingredient types or food items specified by the user as undesirable and to be excluded from the generated dinner menus.

The term “allergens” refers to ingredient types, food components, or substances that may cause an adverse biological reaction in the user and that are to be avoided in the generated dinner menus.

The term “analyze” refers to performing computational processing on condition information, including parsing, validation, categorization, and transformation of the information into structures suitable for generating a prompt sentence.

The term “prompt sentence” refers to a sequence of natural language tokens or structured textual content that encodes instructions and constraints for the generative artificial intelligence model to generate dinner menus and associated ingredient information.

The term “generative artificial intelligence model” refers to a machine-learned model configured to receive input tokens representing a prompt sentence and to output generated tokens representing candidate dinner menus and ingredient information, the model being trained using one or more machine learning techniques.

The term “inference processing” refers to execution of the generative artificial intelligence model on computing hardware to produce output data in response to input data, without performing re-training of the model during such execution.

The term “dinner menus” refers to structured representations of one or more meal plans for a designated time period, including at least identification of dishes and associated ingredient information for each dish.

The term “plurality of days” refers to a time span that includes at least two days and typically a sequence of multiple consecutive days such as a week.

The term “ingredient information” refers to data describing components used in preparing dishes, including at least ingredient names, quantities, and measurement units.

The term “aggregate” refers to perform computational operations that combine and summarize multiple pieces of ingredient information across the dinner menus for the plurality of days.

The term “normalization of ingredient names” refers to processing that maps variant textual expressions of ingredient names, including plural forms, synonyms, and spelling variants, to standardized canonical forms.

The term “conversion of units” refers to computational transformation of quantities from one measurement unit to another, such as from kilograms to grams or from pieces to standardized counts.

The term “summation of quantities” refers to numerical aggregation operations that compute a total quantity for each ingredient by adding quantities of identical or normalized ingredient types across multiple menus.

The term “ingredient-total information” refers to data indicating, for each normalized ingredient, a cumulative quantity required over the plurality of days, including standardized units and normalized ingredient identifiers.

The term “external commerce service” refers to a remote electronic commerce platform or service, accessible via a communication network, that provides product information and order processing functions for physical goods such as food ingredients.

The term “product information” refers to data provided by the external commerce service and associated with goods available for purchase, including at least product identification information, description information, and optionally price information and packaging information.

The term “product identification information” refers to data that uniquely identifies a product in the external commerce service, such as a product code, catalog identifier, or similar unique identifier.

The term “order data” refers to structured digital information that specifies items to be purchased, including at least product identification information and corresponding order quantities, and optionally delivery parameters, payment information, and user identifiers.

The term “application programming interface” refers to a defined communication interface, including protocol specifications and data formats, through which the processor programmatically exchanges order data and related information with the external commerce service.

The term “order to a state immediately before placement” refers to a processing state in which the order data has been generated and transmitted to the external commerce service such that the order is prepared and ready to be finalized upon additional confirmation.

The term “confirmation operations” refers to user actions, performed via the communication terminal, that indicate approval or disapproval of the generated dinner menus and order data, including explicit acceptance or rejection commands.

The term “correction operations” refers to user actions that modify properties of the dinner menus or order data, including changes to dishes, ingredients, quantities, or scheduled delivery parameters.

The term “meal history information” refers to data representing meals previously generated, selected, or consumed by the user, including associated timestamps, dish identifiers, and ingredient usage.

The term “health condition information” refers to data indicating physiological or medical attributes of the user relevant to dietary planning, including but not limited to nutritional requirements, medical restrictions, and physician-recommended dietary guidelines.

The term “learning model” refers to a machine-learned model, which may be the same as or different from the generative artificial intelligence model, configured to perform prediction, classification, or recommendation based on accumulated user-related information.

The term “learning processing” refers to computational procedures that adjust parameters of the learning model using training data, including at least user meal history information, preference information, and health condition information.

The term “optimize” refers to adjust or refine parameters, content, or structure of the prompt sentence or dinner menus so as to better match user-specific conditions, reduce constraint violations, or improve predicted user satisfaction.

The term “input operation information” refers to data representing user interactions with the communication terminal, including touch operations, key inputs, pointer movements, and interaction timing patterns.

The term “voice information” refers to audio data acquired from a microphone associated with the communication terminal, including spoken utterances and acoustic features that may be used to infer user emotion.

The term “image information” refers to visual data acquired from an image capture device associated with the communication terminal, including facial images or body posture images that may be processed for emotion estimation.

The term “emotional state” refers to an inferred psychological or affective condition of the user, such as stress level, happiness, sadness, or boredom, estimated based on input operation information, voice information, or image information.

The term “presentation order” refers to a sequence or arrangement in which dinner menus or related information are displayed to the user on the communication terminal.

The term “presentation expression” refers to stylistic and structural aspects of how the dinner menus and related information are displayed, including layout, emphasis, textual descriptions, and graphical elements.

The term “proposal contents” refers to specific aspects of the dinner menus that are recommended to the user, including dish selection, variation suggestions, and alternative ingredient recommendations.

The term “presentation method of recipes” refers to a set of rules and behaviors controlling how recipes and related information are rendered, ordered, and adapted in the user interface, including dynamic changes based on estimated emotional state.

In one embodiment, a server includes at least one processor, a main memory, a non-volatile storage unit, and a network interface card connected via a system bus. The server executes an operating system and a middleware stack that hosts a plurality of software modules implementing the functions described below. A terminal, such as a smartphone, tablet, or personal computer, includes a processor, a display device, an input device such as a touch panel or keyboard, a microphone and camera in some embodiments, and a wireless or wired communication interface. A user operates the terminal to interact with the server over a packet-switched network.

The server executes an application including a front-end interface module, an API server module, a data management module, a generative AI orchestration module, an ingredient aggregation module, a commerce integration module, a user modeling module, and an emotion estimation and presentation control module. The terminal executes a web browser or a native application that communicates with the API server module of the server.

In one implementation, the server uses a relational database management system such as a general-purpose relational database to store user condition information, recipe records, ingredient mappings, commerce product catalogs, user history data, and model configuration data. The generative AI orchestration module uses a machine learning framework such as a general-purpose tensor-based computation framework or a deep learning framework to execute a generative AI model. The generative AI model is implemented as a transformer-based neural network including an input embedding layer, a positional encoding mechanism, a plurality of self-attention layers, feed-forward networks, and a final output projection layer. The model parameters are stored in a model repository on the non-volatile storage unit and are loaded into memory when inference is performed. The server may employ one or more graphics processing units to accelerate matrix multiplications in the self-attention layers.

The server receives, from the terminal, condition information including at least numerical budget information and attribute information such as disliked ingredients and allergens. The terminal presents input widgets, for example text fields and selection lists, rendered by a browser or a native UI framework, and transmits the user's inputs to the server in a structured data format over a secure transport protocol. The server validates and normalizes the received condition information. For example, the server converts budget values into a canonical currency unit, lowercases ingredient names, and maps known allergens and ingredients to standardized identifiers using a dictionary table stored in the database.

The server stores the normalized condition information in a user conditions table. The server then generates a prompt sentence for the generative AI model by combining constant template text and the normalized condition fields. The server uses string concatenation operations and token-safe escaping of user-provided terms to avoid ambiguities. The prompt sentence is stored in a log table for traceability and later analysis.

In one example, the server generates a prompt sentence such as:

“You are a meal planner. Generate 7 dinner recipes for one week. Conditions: total budget 5000 yen, disliked ingredients: tomato, allergens to avoid: nuts. For each day, output one dinner recipe with a dish name, a list of ingredients with quantities and units, and simple step-by-step cooking instructions. Do not include tomato or any type of nuts in any recipe.”

In another example, the server generates a more structured prompt sentence that constrains the output style:

“You are a professional nutrition-focused meal planner. Under the following constraints, generate 7 daily dinner menus. Constraints: total budget 5000 yen, disliked ingredients: tomato, allergens to avoid: nuts. For each day, provide: (1) a dish name; (2) ingredients in the format ‘ingredient_name: quantity unit’; (3) short cooking steps. Ensure that the sum of estimated ingredient costs for all 7 days does not exceed 5000 yen, and exclude tomato and nuts from all recipes.”

The server supplies the prompt sentence as input to the generative AI model. The generative AI orchestration module first tokenizes the prompt sentence using a predefined tokenizer associated with the transformer model. The tokenizer maps each text token to an integer index, and these indices are converted into embedding vectors. The transformer network propagates these vectors through multiple layers of self-attention and feed-forward computation. Each self-attention layer computes attention weights between all pairs of tokens by performing scaled dot-product operations, followed by softmax normalization, thereby enabling the model to capture long-range dependencies between budget, ingredient constraints, and recipe structure. The model then autoregressively generates output tokens representing the recipes, subject to decoding constraints such as a maximum token length and a nucleus sampling or beam search strategy.

The server receives the generated tokens and converts them back into text using the tokenizer's inverse mapping. The raw generated text typically includes multiple days' recipes separated by line breaks or markers. The server parses the text using a deterministic parser configured with pattern rules, regular expressions, and delimiter recognition logic. For example, the server recognizes headers such as “Day 1:” or “Recipe 1:” and segments the text into daily recipes. For each recipe, the server identifies a dish name line, ingredient lines formatted with an ingredient name and a quantity and unit, and cooking instruction lines. The server converts the parsed content into an internal recipe data structure, such as a list of records each including fields for day index, dish name, ingredient list, and instructions.

The server performs a post-generation constraint check. Specifically, the server compares each ingredient name, after normalization, against the user's disliked ingredients and allergens using case-insensitive string matching and dictionary-based synonym resolution. If a prohibited ingredient is detected, the server marks the corresponding recipe as invalid and may either discard it, request regeneration for that day with a revised prompt sentence that explicitly excludes the offending ingredient, or automatically substitute a recommended alternative ingredient from a substitution table. This post-processing is implemented at the software level in the server and reduces dependency on perfect behavior of the generative AI model, thus improving reliability.

The server then aggregates the ingredient information across all generated daily recipes. The ingredient aggregation module iterates through each recipe's ingredient list. For each ingredient entry, the server normalizes the name using a canonical mapping table. The server also converts units into a standardized base unit, for example converting kilograms, grams, and milligrams into grams; or converting “piece” and “pack” into a standard item count using packaging metadata. The server maintains an associative data structure, such as a hash map keyed by ingredient identifier, and accumulates total quantities by performing numerical addition. This aggregation yields ingredient-total information that specifies, for each normalized ingredient, a total quantity required for the plurality of days.

The server associates each normalized ingredient with one or more purchasable products available from an external commerce service. The commerce integration module queries a product catalog table that stores product identifiers, packaging units, and mapping rules between ingredients and products. Where necessary, the server calculates how many product units are needed to cover the aggregated quantity by dividing the total quantity by the quantity per product unit and rounding up. The server constructs order data including product identification information, order quantities, and optional metadata such as delivery time preferences and user identifiers.

The server transmits the order data to an application programming interface of the external commerce service using a structured request format over a secure protocol. The server does not immediately finalize payment or commit the order in some embodiments; instead, the order is prepared to a state immediately before placement, enabling the user to confirm or modify the contents. The commerce service can respond with provisional order identifiers, item availability information, and price estimates, which the server stores together with the recipe data in the database.

The server sends the final recipes and the associated order information to the terminal. The terminal renders a user interface where the user can browse the proposed weekly dinner menus, view ingredient lists, and examine the derived shopping list with product-level details. The terminal allows the user to perform confirmation operations such as accepting or discarding individual recipes, and correction operations such as adjusting ingredient quantities or replacing a dish. The terminal transmits these edits back to the server. The server updates the recipe records, recomputes ingredient-total information as needed, and regenerates the order data. This closed-loop interaction allows the server to preserve consistency between the displayed menus, aggregated ingredients, and prepared orders.

The server additionally executes a user modeling module that uses a learning model, which may be separate from the generative AI model, to capture user-specific patterns over time. The server stores user meal history information, including identifiers of selected recipes, timestamps, and feedback signals such as whether an order was completed or canceled. The server also stores preference information and health condition information when provided. The learning model is implemented as a neural network, such as a feed-forward network or recurrent network, or as a gradient boosted decision tree model. The server trains the learning model offline or periodically by minimizing a loss function that measures prediction error between the model's outputs (e.g., predicted user satisfaction or adherence to constraints) and observed outcomes. The loss function may be a cross-entropy loss for classification or a mean squared error for regression. The server performs weight updates using gradient-based optimization methods such as stochastic gradient descent or Adam, computing gradients via automatic differentiation provided by the underlying machine learning framework.

The server uses the trained learning model to adaptively modify prompt sentences and generation parameters. For example, if the model predicts that a certain user is highly price-sensitive, the server increases the weight of budget constraints in the prompt sentence, explicitly requiring lower-cost ingredients or simpler dishes. If the model predicts a high preference for certain cuisines or cooking times, the server inserts those preferences into the prompt sentence. As a result, the generative AI model receives prompt sentences that are better aligned with each user's learned profile, which reduces the number of post-generation corrections and lowers re-generation frequency. This improves computational efficiency by reducing unnecessary calls to the generative AI model and minimizing redundant parsing and aggregation steps.

In another embodiment, the server estimates a user's emotional state to further control recipe presentation behavior. The terminal acquires input operation information, such as rapid consecutive taps, prolonged pauses, or repeated back-and-forth navigation, and may optionally send voice information or image information captured by built-in sensors. The server processes voice information using a speech feature extractor that computes mel-frequency cepstral coefficients or similar features, and processes image information using a convolutional neural network trained to detect facial expressions. The emotion estimation model outputs a probability distribution over discrete emotional states. The server uses this output as a control signal for the presentation control module. For example, when the user appears stressed or fatigued, the server instructs the terminal to present fewer choices at once, reorder menus to show simpler recipes first, and emphasize reassuring textual descriptions. By integrating emotion estimation into the server's presentation logic, the system reduces cognitive load and network chatter associated with repeated user queries and page reloads.

These configurations provide technical improvements beyond mere automation of human tasks. The tight coupling between the generative AI orchestration module, the deterministic parsing and aggregation algorithms, the commerce integration module, and the adaptive user modeling leads to reduced end-to-end latency and more efficient use of hardware resources. Specifically, the server decreases unnecessary re-computation by actively shaping the prompt sentence based on learned user profiles, thereby reducing the average number of generations required to obtain an acceptable weekly plan. The structured parsing and ingredient aggregation minimize memory usage and bandwidth by representing recipes in compact, normalized data structures instead of leaving them as unstructured text throughout the pipeline. The commerce integration with unit conversion and product mapping at the server reduces the number of back-and-forth communications with external services because the server batches ingredient demands across multiple days into a single optimized order. Further, by executing neural network inference on dedicated hardware accelerators and pre-loading model weights into memory, the server improves response times compared to ad hoc or manual handling of recipe planning.

In one variation, the server uses different generative AI models for different categories of users, such as a smaller model for users with limited budgets and a larger model for users who require highly diverse menus. The server may select a model based on the number of ingredients, history length, or hardware load. In another variation, the ingredient aggregation module supports multiple unit systems and automatically selects appropriate units based on locale information of the terminal. In yet another embodiment, the server caches intermediate results such as normalized ingredient lists for frequently generated recipes and reuses them when similar prompts are issued, further improving computational efficiency.

In all of these embodiments, the server, the terminal, and the user cooperate through well-defined data structures, concrete algorithmic steps, and hardware-accelerated model execution. The generative AI model is not used as a black box but is driven by carefully constructed prompt sentences, post-processed by deterministic parsing, and combined with structured aggregation and commerce integration logic. This architecture provides a technical solution that enhances computer functionality in the domains of data management, inference efficiency, and network communication, and enables reliable generation, structuring, and ordering of ingredients for multi-day dinner menus in a manner that could not be practically achieved by manual human procedures alone.

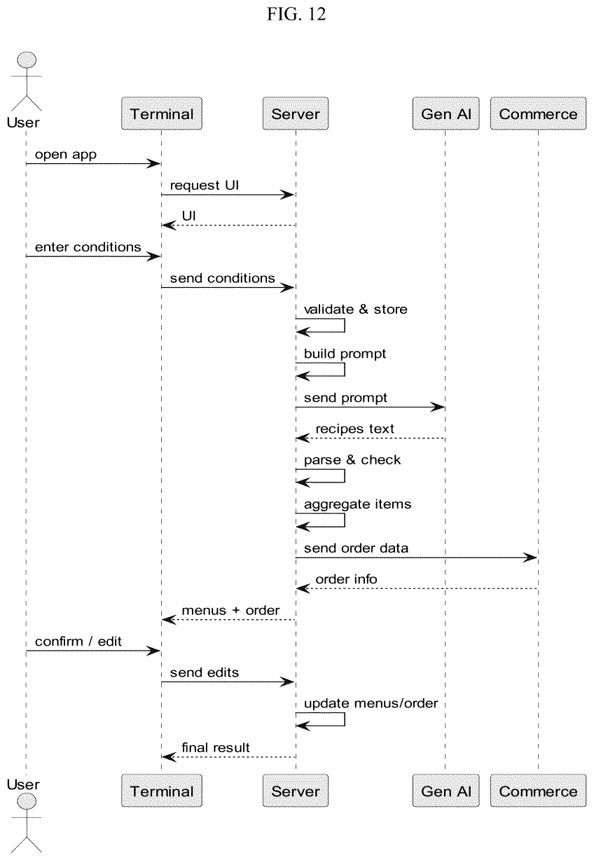

The following describes the processing flow using FIG. 12.

### Step 1

The user operates the terminal to start a meal-planning session. The terminal displays an input screen including fields for budget, disliked ingredients, and allergens. The input of this step is a user action (screen open request), and the output is a rendered user interface on the terminal. The terminal sends a screen request to the server, the server returns layout and configuration data, and the terminal renders text boxes, selection lists, and buttons according to that data.

### Step 2

The user inputs condition information via the terminal. The input of this step is the displayed input screen, and the output is a set of raw condition values (budget text, disliked ingredient strings, allergen strings) held in the terminal's memory. The terminal performs client-side validation by executing comparison and pattern-matching operations on the entered values, such as checking that the budget contains only digits and that text fields do not exceed predefined length limits.

### Step 3

The terminal transmits normalized condition information to the server. The input of this step is the validated raw condition values, and the output is a structured condition object sent over the network. The terminal converts the values into a structured representation (for example, a key-value map), encodes it into a request body, and sends it to the server via a network interface. The terminal also attaches a user identifier or session token so that the server can associate the condition information with the correct user.

### Step 4

The server receives and validates the condition information. The input of this step is the structured condition object from the terminal, and the output is a normalized condition record stored in a database. The server parses the request body, checks that the budget is within an allowable range by numerical comparison, and converts ingredient names and allergen names to lowercase. The server looks up each ingredient and allergen in a reference table to map them to canonical identifiers. The server then executes an insertion or update operation in a user conditions table to persist the normalized condition information.

### Step 5

The server generates a prompt sentence for the generative AI model. The input of this step is the normalized condition record retrieved from the database, and the output is a prompt sentence in natural language. The server concatenates template text with the budget value, disliked ingredient list, and allergen list by using string operations. The server joins multiple ingredient names into a comma-separated sequence and embeds that sequence into the template. For example, the server constructs: “You are a meal planner. Generate 7 dinner recipes for one week. Conditions: total budget 5000 yen, disliked ingredients: tomato, allergens to avoid: nuts. For each day, output one dinner recipe with a dish name, a list of ingredients with quantities and units, and simple step-by-step cooking instructions. Do not include tomato or any type of nuts in any recipe.”

### Step 6

The server executes inference on the generative AI model using the prompt sentence. The input of this step is the prompt sentence, and the output is generated recipe text representing multiple days of dinner menus. The server invokes a tokenizer to transform the prompt sentence into a sequence of integer token IDs, loads model parameters into memory, and feeds the token sequence into a transformer-based neural network. The neural network performs matrix multiplications, attention weight calculations, and non-linear activations across multiple layers to compute output token probabilities. The server decodes these probabilities into tokens using an autoregressive decoding strategy and finally converts the tokens back into human-readable text.

### Step 7

The server parses the generated recipe text into structured recipe data. The input of this step is the raw generated text from the generative AI model, and the output is a collection of structured recipe records. The server scans the text line by line, detects separators such as “Day 1:” or blank lines, and segments the text into individual daily recipes. The server applies pattern-matching rules to identify dish names, ingredient lines, and instruction lines. For each ingredient line, the server isolates the ingredient name, numeric quantity, and unit by splitting on delimiters and converting numeric substrings into numeric types. The server builds an internal data object for each day containing a dish name, a list of ingredient objects, and a set of instructions.

### Step 8

The server checks the structured recipe data against the user's restrictions. The input of this step is the structured recipe records and the normalized condition record (disliked ingredients and allergens), and the output is a validated and possibly corrected set of recipes. The server normalizes each ingredient name using a dictionary and compares it to the lists of disliked ingredients and allergens using case-insensitive string comparison. If a restricted ingredient is found, the server flags the recipe and either removes it, replaces the ingredient using a substitution table, or regenerates a recipe for that day with a revised prompt sentence that explicitly reinforces the exclusion. The server thereby enforces constraints through conditional branching and table lookups.

### Step 9

The server aggregates ingredients across all valid recipes. The input of this step is the validated recipe records, and the output is ingredient-total information representing total quantities per normalized ingredient. The server iterates over each recipe's ingredient list and performs the following data processing: the server maps each ingredient name to a canonical identifier, converts the quantity into a standard unit by multiplying by appropriate conversion factors, and updates a hash map keyed by ingredient identifier. For each ingredient identifier, the server adds the quantity from the current recipe to the existing total using numeric addition. After processing all recipes, the server produces a list of ingredients with aggregated quantities.

### Step 10

The server maps aggregated ingredients to products in an external commerce service and generates order data. The input of this step is the ingredient-total information and a product catalog table, and the output is structured order data including product identifiers and order quantities. The server queries the product catalog for each ingredient identifier, retrieves product unit size and product identifier, and computes the required number of product units by dividing the total quantity by the unit size and applying a ceiling operation. The server constructs an order item list by adding entries containing product identifiers and computed quantities. The server then assembles these order items with user delivery settings into an order data object.

### Step 11

The server transmits the order data to an external commerce service and receives a provisional response. The input of this step is the order data, and the output is a response object containing at least an order identifier, price information, and availability information. The server formats the order data according to the commerce service's application programming interface specification, encodes it in a request message, and sends it through the network interface using a secure protocol. The external service returns a response; the server parses the response, extracts identifiers and pricing, and stores them in an orders table associated with the user.

### Step 12

The server sends the recipes and order information to the terminal for display. The input of this step is the validated recipe records and the response object from the commerce service, and the output is a structured payload delivered to the terminal. The server bundles the recipes, aggregated ingredient list, and provisional order summary into a single response and transmits it to the terminal. The terminal decodes the response, builds user interface components for each day's recipe, and displays a shopping summary, showing item names, quantities, and total cost.

### Step 13

The user reviews, confirms, or corrects the recipes and order information via the terminal. The input of this step is the displayed user interface containing recipes and order details, and the output is a set of user confirmation and correction commands. The user may tap buttons to accept or reject specific recipes, modify ingredient quantities using sliders or text fields, or change delivery details. The terminal captures these operations as structured edit commands and transmits them to the server.

### Step 14

The server processes user corrections and updates recipe and order data. The input of this step is the set of edit commands from the terminal, and the output is an updated recipe collection and updated order data. The server interprets each edit command, modifies the corresponding recipe records or ingredient-total information, and recalculates affected aggregated quantities. The server then recomputes product unit counts and updates the order data accordingly. The server persists the updated state in the database and may retransmit the updated summary to the terminal for final confirmation.

### Step 15

The server logs interaction data and updates user models. The input of this step is the final accepted recipes, the user's edit history, and order completion status, and the output is updated model training data and, optionally, updated model parameters. The server writes interaction events into a history table, including which recipes were accepted, what corrections were made, and whether the order was finalized. The server periodically feeds this history into a learning model, computes loss values between predicted and observed behaviors, and adjusts model weights using gradient-based optimization. The updated model parameters change the way the server constructs future prompt sentences and sets generation parameters, which in turn affects future recipe generation behavior.

### Step 16

The terminal notifies the user of the final state and waits for real-world delivery. The input of this step is the final confirmation result from the server and, optionally, delivery schedule information from the commerce service, and the output is a confirmation screen and status indicators on the terminal. The terminal displays the finalized weekly menu, the finalized list of ingredients and products, and the scheduled delivery time. The user can then close the application or monitor status updates.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented recipe and meal-planning systems typically operate as thin user-interface layers over static recipe databases or simple rule-based engines. Such systems generally retrieve predefined recipes based on keyword searches, fixed dietary tags, or simple filtering rules, and may present shopping lists that are directly derived from the selected recipes. These approaches suffer from several technical limitations.

First, conventional systems lack an integrated, machine-executable mechanism for transforming heterogeneous user constraints—such as budget ranges, disfavored ingredients, and allergens—into a structured representation that can be consistently consumed by downstream computational components. As a result, the server side often performs ad hoc query composition and repetitive rule evaluation, which imposes unnecessary processing overhead, increases memory access operations, and leads to inefficient use of processing resources.

Second, existing systems do not tightly couple generative models with deterministic nutritional analysis and optimization pipelines. In typical architectures, any generative or recommendation function is invoked in isolation, and its output is either presented directly to the user or manually post-processed. The server does not perform a closed-loop adjustment in which generated recipes are converted into structured data, subjected to machine-executable nutrient calculations using an external nutrient-information store, algorithmically compared to target intake profiles, and then automatically modified and iterated. This lack of a feedback loop leads to suboptimal use of computational resources, because the generative model may repeatedly generate recipes that do not satisfy the nutritional criteria, thereby requiring repeated user interaction and redundant computation.

Third, conventional systems do not provide a unified server-side workflow that propagates optimized, machine-readable recipe structures into an electronic commerce interface in a format that is directly consumable by the electronic transaction apparatus. In many cases, shopping lists are prepared on the client side or as unstructured text, requiring additional parsing, manual mapping to products, and user input. This results in increased network traffic, redundant data transformation steps, and higher latency, as the system must repeatedly synchronize semi-structured data between the user terminal, the server, and external commerce platforms.

Fourth, although some systems may attempt to capture user preferences over time, they typically treat such information as auxiliary metadata rather than as a first-class computational input that systematically modifies how prompts are constructed for generative models and how structured recipe data are algorithmically adjusted. Consequently, these systems fail to exploit the full computational benefits of learning-based personalization on the server side, and instead rely on repetitive filtering or manual tuning, which again increases processing workload and decreases throughput.

Fifth, conventional architectures do not utilize user interaction signals or biometric information to dynamically modify system-generated content at the prompt-construction level. As a result, the server cannot adapt generation conditions or presentation styles in response to user emotional state in a machine-efficient manner, and must instead rely on static templates or client-side logic. This leads to inefficient utilization of server-side processing cycles, as the same types of content are generated and transmitted regardless of user state, potentially requiring repeated interactions and re-generation of content.

Accordingly, there is a need for an improved server-centric system and method that: (i) converts heterogeneous user constraints into structured meal-planning conditions in a form suitable for low-overhead computational processing; (ii) generates recipes by interacting with a generative AI model through explicitly structured prompt sentences; (iii) automatically parses generated text into machine-usable structured data; (iv) performs deterministic nutrient computation and automatic recipe adjustment in a closed computational loop; (v) prepares machine-readable purchase-candidate data for an electronic transaction apparatus; and (vi) optionally leverages learned user preference patterns and user emotional state to modify prompt generation and data adjustment logic. Such improvements are required to reduce redundant computation, improve memory and network utilization, lower server-side latency, and provide a more technically efficient pipeline from user input to optimized meal plans and order-ready ingredient representations.

The specific processing by the specific processing unit 290 of the data processing device 12 in Example 2 is realized by the following means.

The present invention provides a server comprising a processor and a communication interface, the processor being configured to execute instructions that cause the server to: receive, via a communication network from a user terminal, budget information, preference information, and allergen-avoidance information as heterogeneous user constraint data; normalize and analyze the heterogeneous user constraint data to extract standardized meal-planning conditions in a structured internal representation; generate, based on the structured meal-planning conditions and stored nutritional-target information, a machine-readable prompt sentence that encodes explicit constraints for a generative AI model, and transmit the prompt sentence to the generative AI model; receive, from the generative AI model, text data representing cooking procedure information for meals for a plurality of days; parse the text data into structured recipe data including meal information and ingredient information for each day, and store the structured recipe data in a data storage device; acquire nutrient data from a nutrient-information storage apparatus based on the ingredient information and calculate, by numeric computation, nutrient amounts for each meal and for a predetermined period in total; compare the calculated nutrient amounts to predetermined nutrient-intake criteria, and automatically adjust the structured recipe data by algorithmically changing at least one of a kind and a quantity of ingredients so that a nutrient-intake balance falls within a target range, and, when necessary, construct and transmit an additional prompt sentence comprising adjustment information to the generative AI model and update the structured recipe data in accordance with an adjusted output; aggregate, based on the adjusted structured recipe data, ingredient requirements over the predetermined period, generalize the ingredients into merchandise information, and generate purchase-candidate data including the merchandise information and quantity information according to an interface specification of an electronic transaction apparatus, the purchase-candidate data being formatted for direct consumption by the electronic transaction apparatus to establish a purchase-candidate state immediately before ordering; and generate display data for transmission to the user terminal, the display data including at least the structured recipe data, calculated nutrient amounts, and identification information of the purchase-candidate state, such that the user terminal can present meal-planning information and purchase-candidate information without additional parsing. This enables the server to implement a technically integrated, end-to-end pipeline that transforms heterogeneous user constraints into structured prompts, leverages a generative AI model for multi-day meal generation, performs deterministic nutrient computation and automatic recipe optimization in a closed computational loop, and outputs machine-ready purchase-candidate data for an electronic transaction apparatus, thereby reducing redundant processing, improving resource utilization, and decreasing latency in generating and provisioning optimized meal plans and associated purchase data.

The term “user terminal” refers to an information processing device operated by a user, including but not limited to a mobile device, a tablet device, or a personal computer, that communicates with the server via a communication network and presents user interface screens for inputting and viewing data.

The term “communication network” refers to a wired or wireless data transmission infrastructure, including but not limited to the Internet, local area networks, cellular networks, and combinations thereof, through which data is exchanged between the server, the user terminal, and external systems.

The term “budget information” refers to data indicating a monetary constraint or range associated with acquisition of ingredients or meals over a predetermined period, including but not limited to a total budget value, a per-day budget value, or a per-meal budget value.

The term “preference information” refers to data indicating user-specific food-related preferences, including but not limited to disliked ingredients, preferred ingredients, cuisine types, preparation methods, or any other qualitative or quantitative expression of meal preferences.

The term “allergen-avoidance information” refers to data specifying substances or food categories that a user wishes to exclude from meals in order to avoid allergic reactions or other adverse health effects.

The term “heterogeneous user constraint data” refers to a collection of multiple different kinds of user-provided information, including but not limited to budget information, preference information, and allergen-avoidance information, which differ in data type, format, or semantic content.

The term “meal-planning conditions” refers to internal constraint data derived from heterogeneous user constraint data, expressed in a structured, normalized form suitable for programmatic processing to generate one or more meal plans.

The term “nutritional-target information” refers to data defining one or more desired nutritional goals, including but not limited to target total energy intake, target ranges of macronutrients, or other nutrient-related limits or objectives for a predetermined period.

The term “generative AI model” refers to a computational model using machine learning techniques, such as a neural network or transformer-based architecture, that is configured to generate text or other content in response to an input prompt sentence specifying desired conditions or constraints.

The term “prompt sentence” refers to a machine-readable text string or sequence of tokens transmitted to a generative AI model, the text string encoding explicit instructions, constraints, or conditions for generating one or more outputs.

The term “cooking procedure information” refers to data describing one or more meals, including but not limited to recipe titles, ingredient lists with quantities, and ordered preparation steps required to produce the meals.

The term “structured recipe data” refers to a machine-readable representation of cooking procedure information, organized into data fields such as meal identifiers, ingredient identifiers, quantities, and preparation steps, suitable for storage in a database and programmatic manipulation.

The term “meal information” refers to structured data describing a specific meal, including but not limited to a meal identifier, a recipe title, and associated preparation steps.

The term “ingredient information” refers to structured data describing ingredients used in one or more meals, including but not limited to ingredient names, quantities, measurement units, and associations with specific meals.

The term “nutrient-information storage apparatus” refers to a logical or physical data storage resource, including but not limited to a database, a file system, or an external service, that stores nutrient data for different food items or ingredients.

The term “nutrient data” refers to information describing nutritional properties of food items or ingredients, including but not limited to energy values and quantities of macronutrients and micronutrients per unit weight, volume, or serving.

The term “nutrient amounts” refers to numeric values representing quantities of one or more nutrients contained in a meal or meals over a predetermined period, calculated based on ingredient information and nutrient data.

The term “nutrient-intake criteria” refers to threshold values or ranges that define acceptable or target levels of nutrient intake for a user or a predetermined period, including but not limited to minimum or maximum limits for calories, proteins, fats, carbohydrates, or other nutrients.

The term “nutrient-intake balance” refers to a relationship between one or more calculated nutrient amounts and corresponding nutrient-intake criteria, indicating whether the amounts are within, above, or below target ranges.

The term “adjusted structured recipe data” refers to structured recipe data that has been modified by changing at least one of the kind and the quantity of ingredients, based on nutrient-intake criteria or other constraints, to improve or achieve a desired nutrient-intake balance.

The term “merchandise information” refers to data representing goods that can be transacted via an electronic transaction apparatus, including but not limited to product identifiers, generalized ingredient descriptors, packaging units, or other attributes suitable for purchase.

The term “purchase-candidate data” refers to structured data that defines one or more potential purchase items and associated quantities, formatted in accordance with an interface specification of an electronic transaction apparatus and usable to generate a purchase-candidate state immediately before ordering.

The term “electronic transaction apparatus” refers to a computerized system, including but not limited to an e-commerce platform or online marketplace, that is configured to receive purchase-candidate data and process electronic transactions for goods or services.

The term “purchase-candidate state” refers to an electronic state maintained by an electronic transaction apparatus in which one or more items and quantities are prepared for ordering, such as the contents of a shopping cart or order draft, prior to final transaction confirmation.

The term “display data” refers to data formatted for presentation on a user terminal, including but not limited to structured recipe data, nutrient amounts, and purchase-related identifiers, which can be rendered by a user interface to visually present information to a user.

The term “past input information” refers to records of previous user-provided data, including but not limited to historical budget information, preference information, and allergen-avoidance information stored by the server over time.

The term “selection history” refers to records of user choices or interactions with presented content, including but not limited to selected recipes, accepted meal plans, or confirmed purchases, used by the server for learning or personalization.

The term “tendencies of a user's meal preferences and health conditions” refers to patterns inferred from past input information and selection history, indicating typical user choices or constraints regarding ingredients, meal types, nutritional limits, or health-related requirements.

The term “operation-history information” refers to data describing user interactions with the user terminal, including but not limited to input frequency, navigation behavior, pointer movements, and selection patterns, which can be used to infer user state.

The term “biometric information” refers to physiological or behavioral measurements associated with the user, including but not limited to heart rate, facial expression data, or voice characteristics, which can be used to estimate an emotional state.

The term “emotional state” refers to a mental or affective condition of the user, such as stress, calmness, excitement, or boredom, inferred from operation-history information or biometric information.

The term “style of the prompt sentence” refers to linguistic characteristics of a prompt sentence, including but not limited to tone, level of detail, politeness, or narrative structure, which can be varied while directing the generative AI model.

The term “output condition of the prompt sentence” refers to constraints or parameters specified by a prompt sentence that control properties of the generative AI model output, including but not limited to length, format, level of complexity, or content focus.

The term “meal-planning information” refers to information presented to the user that summarizes generated or adjusted meals over a predetermined period, including but not limited to recipe listings, scheduled meal assignments, and associated nutritional summaries.

The term “purchase-candidate information” refers to information presented to the user identifying items included in the purchase-candidate state, including but not limited to ingredient names, quantities, and links or identifiers for completing purchase transactions.

In one embodiment, a server includes a processor, a main memory, a non-volatile storage device, and a communication interface. The server is connected to a communication network and communicates with one or more user terminals and one or more electronic transaction apparatuses. The user terminal includes, for example, a smartphone, a tablet device, or a personal computer, which executes a web browser such as a hypertext markup language (HTML) compliant browser and presents screens for user interaction.

The server uses an operating system such as a general-purpose server operating system and executes application software implemented with a server-side framework such as a scripting-based web framework or a compiled-language web framework. The server stores structured data in a relational database management system such as a structured query language (SQL) based database. The server accesses external services, such as a nutrient-information storage service and a generative AI model service, via application programming interfaces (APIs) over the communication network.

The user uses the terminal to access a web application provided by the server. The terminal displays input fields for budget information, preference information, and allergen-avoidance information. The user uses the terminal to enter budget ranges, lists of disliked ingredients, and allergen items. The terminal converts the entered values into a structured request, for example in a JavaScript object notation (JSON)-compatible format, and transmits the request to the server via the communication interface.

The server receives the heterogeneous user constraint data and stores it in memory in the form of typed data structures. The server uses a data-normalization module to convert free-text entries and enumerated fields into a canonical internal representation, such as normalized ingredient identifiers, numeric budget values expressed in a unified currency unit, and allergen codes. The server uses dictionaries and synonym tables stored in the database to map user-entered ingredient names to canonical ingredient identifiers. By converting diverse inputs into normalized codes and scalar values, the server reduces complexity of later computations and reduces the number of string-comparison operations during subsequent processing.

The server converts the normalized user constraint data into structured meal-planning conditions. The server uses a rule engine, implemented as a deterministically evaluated decision table, to classify constraints into categories such as budget constraints, exclusion constraints, and nutritional constraints. The server stores the meal-planning conditions as an internal record with fields including a maximum budget per period, sets of disallowed ingredient identifiers, and sets of disallowed allergen identifiers. This structured representation reduces the need to repeatedly parse natural-language conditions and enables the processor to perform efficient, indexed lookups when constructing prompts and analyzing recipes.

The server uses a generative AI model to obtain multi-day cooking procedure information. In one embodiment, the generative AI model is a transformer-based neural network trained on large-scale text data representing recipes and cooking instructions. The server communicates with the generative AI model via an API. The server does not treat the model as a black box, but instead configures the model with a specified architecture and hyperparameters. For example, the model includes an embedding layer that maps text tokens to vector representations, multiple self-attention layers that compute attention scores over token sequences, and feed-forward layers that apply non-linear transformations. The model is trained by minimizing a cross-entropy loss function over next-token prediction, using an optimizer such as stochastic gradient descent with adaptive moment estimation. During inference, the model generates output tokens based on a temperature parameter, a top-k or top-p sampling scheme, and a maximum token length specified by the server.

The server uses a prompt-construction module to generate a prompt sentence for the generative AI model. The server combines a base template, fixed system-level instructions, and variable segments derived from the meal-planning conditions. The server constructs the prompt sentence in a deterministic manner, using string concatenation, placeholder substitution, and explicit constraint encoding. The server represents the prompt sentence in text format suitable for input to the generative AI model.

For example, the server generates a prompt sentence such as:

“You are a professional nutritionist and chef. Generate 7 dinner recipes for one week that are high in protein and low in fat. The daily target energy intake is 2000 kilocalories. The weekly budget for ingredients is 5000 units of currency. Avoid the following ingredients that the user dislikes: green pepper, blue cheese. Completely exclude the following allergens: eggs, milk, peanuts. For each recipe, output the recipe title, a list of ingredients with quantities, and step-by-step cooking instructions in English.”

In another example, the server generates a prompt sentence such as:

“Generate 7 cost-effective dinner recipes for one week for a single adult. The weekly food budget is 5,000 units of currency. Each day's total energy should be around 2000 kilocalories, with high protein and moderate carbohydrates. Avoid eggs and milk due to allergy, and avoid mushrooms and cilantro due to user preference. Use common ingredients that can be purchased on a general online marketplace. For each recipe, list the ingredients, approximate quantities, and simple cooking instructions. Try to reuse ingredients across recipes to reduce total cost.”

The server transmits the prompt sentence to the generative AI model and receives generated text representing multiple recipes. The server stores the raw text response into memory and initiates a structured-parsing process. The server uses a text-parsing module that applies regular expressions and tokenization to segment the text by recipe, day, ingredient list, and preparation steps. The server maps each identified ingredient phrase to a canonical ingredient identifier via the same normalization dictionary used for user inputs. The server thus obtains structured recipe data including, for each recipe, a recipe identifier, a list of ingredient identifiers with quantities and units, and an ordered list of cooking steps. The server stores this structured recipe data in one or more relational tables, such as a “recipes” table, an “ingredients” table, and a “recipe_ingredients” mapping table.

The server then performs a deterministic nutrient calculation for each recipe. The server accesses a nutrient-information storage apparatus, which may be implemented as an internal or external database containing per-100-gram nutrient data for each canonical ingredient identifier. The server uses indexed queries to retrieve nutrient vectors storing energy, protein, fat, carbohydrate, and optionally other nutrient components for each ingredient. The server multiplies each nutrient vector by a scalar quantity derived from the recipe ingredient amount (converted into grams or other uniform units), and sums vectors across all ingredients in the recipe. This vectorized computation is implemented in numerical routines that operate on arrays, thereby improving cache locality and reducing repeated database lookups. The server thus computes nutrient amounts for each recipe and aggregates those amounts over the predetermined period (for example, one week).

The server compares the calculated nutrient amounts to stored nutrient-intake criteria. The server holds recommended daily intake values and allowable ranges for major nutrients in a configuration table. The server calculates difference values and ratios, such as the difference between target and actual caloric intake, and the ratio of protein-derived energy to total energy. The server marks recipes or days that deviate beyond configured thresholds. The server stores the comparison results as evaluation flags or scores associated with each recipe record.

The server performs automatic adjustment of the structured recipe data based on the comparison results. The server uses a rule-based adjustment engine that modifies ingredient quantities and kinds according to pre-defined algorithmic rules. For example, when a caloric value exceeds a threshold, the server reduces quantities of high-calorie ingredients by a proportion and optionally replaces certain ingredients, such as substituting lower-fat proteins for higher-fat proteins. The server consults the nutrient-information storage apparatus to identify candidate ingredient replacements that satisfy nutrient constraints and do not appear in the user's disallowed or allergen lists. The server applies a constraint solver that ensures that each adjusted recipe still provides a minimum amount of protein while meeting upper bounds on total calories and fat.

In some embodiments, the server constructs an additional prompt sentence that encodes both the originally generated recipe and desired nutrient adjustments. The server, for example, generates a prompt sentence such as:

“Adjust the following dinner recipe to reduce total calories by 20 percent while keeping protein high and avoiding eggs and peanuts. Keep the overall cooking style similar. Here is the recipe to adjust: [recipe text]. Output the adjusted recipe title, updated ingredients with quantities, and updated step-by-step cooking instructions.”

The server sends this prompt sentence to the generative AI model and receives an adjusted recipe. The server parses and normalizes the adjusted recipe in the same manner as the original, and re-runs the nutrient computation. By integrating this closed-loop adjustment, the server reduces the number of full-plan regenerations and limits network round trips to only those cases requiring refinement, improving computational efficiency and reducing latency.

The server aggregates ingredient requirements across all recipes in the predetermined period. The server sums ingredient quantities by canonical ingredient identifier and converts total quantities into purchase units suited for commerce transactions, such as converting grams into package counts. The server then maps canonical ingredient identifiers to generalized merchandise information that an electronic transaction apparatus can understand, such as product categories, generic names, and typical package sizes. The server prepares purchase-candidate data formatted according to the interface specification of the electronic transaction apparatus. The server transmits this purchase-candidate data to the electronic transaction apparatus to establish a purchase-candidate state, corresponding to a shopping cart or pre-order structure, without requiring manual mapping or input by the user.

The server generates display data for the user terminal. The server composes structured data that includes the recipes, nutrient amounts, and an identifier or link representing the purchase-candidate state. The server transmits this display data to the terminal via the communication network. The terminal renders the recipes and nutrient summaries in a user interface and includes an action control, such as a button, that allows the user to open the purchase-candidate state on the electronic transaction apparatus.

In some embodiments, the server uses past input information and selection history to learn tendencies of a user's meal preferences and health conditions. The server maintains a user-profile model that stores, for example, frequencies of particular ingredients in accepted recipes, distributions of targeted energy levels, and patterns of allergen constraints over time. The server may implement a learning algorithm that uses features such as ingredient occurrence counts, categorical diet patterns, and prior nutrient deviations. The server may train a shallow model such as a logistic regression or a deeper neural model that takes as input a feature vector representing historical user choices and outputs a probability distribution over recipe styles or nutrient profiles. The server then uses this model to modify subsequent prompt sentences by, for example, emphasizing certain cuisines, limiting or expanding cost constraints, or adjusting default nutrient targets. By moving this learning-based adaptation into the prompt-construction and recipe-adjustment stages, the server reduces the number of user corrections required and lowers the amount of data that must be transmitted between the terminal and the server.

In other embodiments, the server estimates a user's emotional state based on operation-history information or biometric information received from the terminal. The server may, for example, receive metrics such as rapid repeated clicks, extended idle times, or sensor-based heart rate data. The server applies an emotional-state classifier, which may be implemented as a machine-learned model or a decision-tree with thresholds. The server then adapts the style and output conditions of the prompt sentence. For instance, when the user is estimated to be stressed, the server encodes in the prompt sentence a requirement for simpler recipes with fewer steps and shorter cooking times. This adaptation reduces wasted computation on complex recipes that are likely to be rejected and improves user acceptance, which in turn reduces repeated generation cycles.

The server architecture provides specific technical improvements over conventional computer systems. By normalizing heterogeneous user constraint data into canonical internal representations, the server reduces parsing overhead and enables the use of indexed lookups rather than repeated string comparisons. By implementing a structured parsing pipeline that converts generative AI output into well-defined recipe records, the server enables direct numeric processing of nutrients and efficient vectorized computation. By employing a closed-loop interaction between deterministic nutrient calculations and generative adjustments, the server avoids repeated full re-generation and reduces network traffic to the generative AI model. Furthermore, by formatting purchase-candidate data directly for consumption by the electronic transaction apparatus, the server eliminates the need for client-side text parsing and manual product matching, thereby reducing client processing load and round-trip communications.

The use of a transformer-based generative AI model with explicit prompt sentences, combined with deterministic normalization, nutrient computation, and rule-based or learned adjustment mechanisms, enables the server to perform operations that are not merely a human mental process executed on a computer. The server carries out numerical vector operations on substantive data structures, optimizes data flow by controlling when and how external models and services are invoked, and arranges data in machine-native formats that reduce resource usage. By modifying prompts and structured recipe data based on learned user tendencies and emotional state, the server further reduces redundant processing and increases the chance that a generated plan will be accepted in fewer iterations. These factors contribute to improved processing speed, lower memory and bandwidth usage, and reduced computational cost across the entire meal-planning and purchasing pipeline.

Various modifications and alternative embodiments are possible. The server may incorporate different database systems, such as document-oriented databases, for storing structured recipe data. The generative AI model may use alternative neural network architectures, such as recurrent neural networks or hybrid transformer-convolutional networks, provided that the server maintains control of prompt construction and parsing logic. The nutrient-information storage apparatus may reside on the same physical machine as the server or be provided as a remote service. The server may also integrate additional constraint dimensions, such as cooking time limits or appliance availability, by introducing further structured fields within the meal-planning conditions and corresponding modifications in the prompt sentences and adjustment rules. The user terminal may be implemented as a native application rather than a browser-based interface, while still using the same display data emitted by the server. In all such variations, the essential technical characteristics remain that the server converts heterogeneous constraints into structured prompts, uses a generative AI model in combination with deterministic computational pipelines, and produces machine-readable purchase-candidate data that is directly usable by an electronic transaction apparatus.

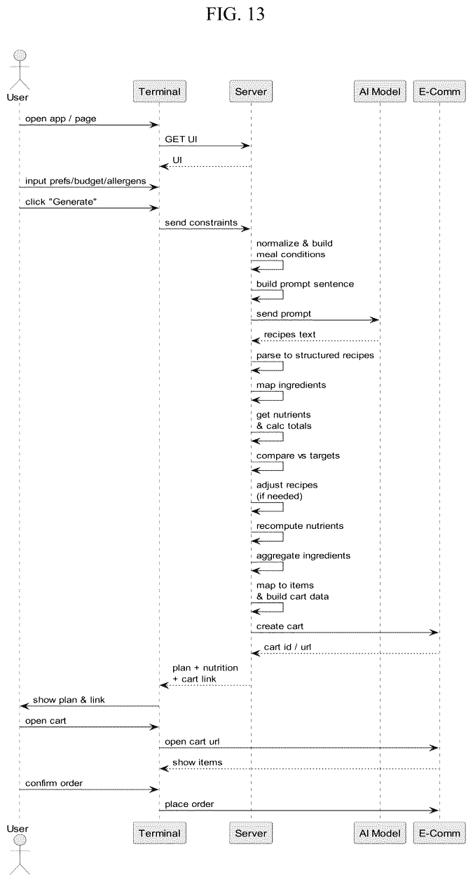

The following describes the processing flow using FIG. 13.

### Step 1

The user uses the terminal to access a web application provided by the server. The terminal sends an access request to the server and receives an HTML/JavaScript page that includes input fields for budget information, preference information, and allergen-avoidance information. As input, the terminal receives the user's manual operations, such as typing values and selecting options, and as output, the terminal generates structured form data stored temporarily in the browser's memory.

### Step 2

The user operates the terminal to enter budget information, lists of disliked ingredients, and allergen-avoidance items into the displayed input fields. The terminal takes, as input, the keystrokes and touch events, converts them into text strings or selected values, and updates the form fields. As output, the terminal holds a set of raw user parameters, such as budget text, preference text, and allergen text, in its document object model (DOM) and JavaScript variables.

### Step 3

The user operates the terminal to trigger a submission action, for example by pressing a “Generate Meal Plan” button. The terminal executes client-side JavaScript, reads the values from the form fields as input, and constructs a request object that includes budget information, preference information, and allergen-avoidance information as key-value pairs. As output, the terminal produces a serialized request payload and transmits it via an HTTPS POST request to the server.

### Step 4

The server receives the HTTPS request via the communication interface. The server uses a web framework to parse the request body and extract the budget information, preference information, and allergen-avoidance information as input parameters. The server validates these parameters by checking data types, required fields, and length constraints. As output, the server produces cleaned and validated user constraint data, or, in the case of invalid data, generates an error response.

### Step 5

The server converts the validated user constraint data into normalized internal representations. The server takes, as input, raw text values such as “5,000 yen,” “green pepper,” and “peanuts,” and applies normalization routines, including unit conversion, trimming, lowercasing, and dictionary-based synonym mapping. The server uses ingredient dictionaries and allergen code tables to map text strings to canonical ingredient identifiers and allergen identifiers. As output, the server produces a structured constraint record that includes numeric budget values, sets of ingredient identifiers to avoid, and sets of allergen identifiers to exclude.

### Step 6

The server derives meal-planning conditions from the normalized constraint record. The server takes, as input, numeric budgets, disallowed ingredient sets, and allergen sets, and applies rule-based logic to classify constraints into categories such as budget constraint, exclusion constraint, and nutritional target. The server may also add default nutritional-target information if not explicitly provided. As output, the server generates a meal-planning condition object that encodes maximum budget per period, excluded ingredient identifiers, excluded allergen identifiers, and target nutrient ranges.

### Step 7

The server constructs a prompt sentence for the generative AI model based on the meal-planning conditions. The server takes, as input, the meal-planning condition object and a base prompt template stored in configuration, and performs string concatenation and placeholder substitution. The server embeds the budget, excluded ingredients, excluded allergens, and nutritional targets into natural language instructions. As output, the server produces a complete prompt sentence, such as: “You are a professional nutritionist and chef. Generate 7 dinner recipes for one week that are high in protein and low in fat. The daily target energy intake is 2000 kilocalories. The weekly budget for ingredients is 5000 units of currency. Avoid the following ingredients that the user dislikes: green pepper, blue cheese. Completely exclude the following allergens: eggs, milk, peanuts.”

### Step 8

The server transmits the prompt sentence to the generative AI model and receives generated recipe text. The server takes, as input, the constructed prompt sentence and wraps it in an API request with parameters such as model name, maximum token length, and temperature. The server sends this request via HTTPS to an external generative AI service. The generative AI model executes internal neural-network computations and returns generated text as a response. As output, the server obtains a text block that contains multiple recipes with titles, ingredient lists, and cooking instructions.

### Step 9

The server parses the generated recipe text into structured recipe data. The server takes, as input, the text block from the generative AI model and applies text-processing algorithms, such as tokenization, regular expressions, and delimiter detection, to identify individual recipes, ingredient sections, and instruction sections. The server splits the text by day labels or headings, extracts ingredient lines, and separates quantities, units, and ingredient names. As output, the server produces structured recipe records that include recipe identifiers, ingredient lists with quantities and units, and ordered cooking steps.

### Step 10

The server normalizes ingredient names in the structured recipe data. The server takes, as input, ingredient names such as “chicken breast,” “olive oil,” and “brown rice,” and consults internal dictionaries and mapping tables that associate text labels with canonical ingredient identifiers. The server performs lowercasing, trimming, and synonym replacement to match against dictionary entries. As output, the server generates normalized ingredient lists for each recipe, where each item includes a canonical ingredient identifier, a numeric quantity in a base unit (for example grams), and a standardized unit.

### Step 11

The server retrieves nutrient data for each normalized ingredient and computes nutrient amounts. The server takes, as input, the normalized ingredient lists with identifiers and quantities, and queries a nutrient-information storage apparatus, such as a nutrient database, using the ingredient identifiers as keys. The server receives nutrient vectors (for example calories, protein, fat, carbohydrates per 100 grams) and converts them to per-quantity values by multiplying by quantity/100. The server sums the nutrient vectors across all ingredients in a recipe to obtain total nutrient amounts for that recipe. As output, the server stores computed nutrient amounts per recipe and aggregated nutrient amounts for the full planning period.

### Step 12

The server compares computed nutrient amounts against nutrient-intake criteria. The server takes, as input, the per-recipe and aggregate nutrient amounts and the stored nutrient-intake criteria, such as target daily calories and allowable macronutrient ranges. The server computes differences and ratios, such as actual minus target calories and percentage of calories from fat. The server evaluates whether each recipe or day falls within allowable ranges by comparing numeric values to thresholds. As output, the server generates evaluation results, including flags marking recipes as acceptable, high-calorie, low-protein, or otherwise unbalanced.

### Step 13

The server identifies recipes that require adjustment and applies deterministic adjustment rules. The server takes, as input, the evaluation results and the structured recipe data. For each recipe flagged as unbalanced, the server analyzes which nutrient constraints are violated (for example too many calories or too little protein) and selects candidate ingredients for modification. The server reduces quantities of high-calorie or high-fat ingredients and increases quantities of low-fat, high-protein ingredients according to rule tables and proportional scaling formulas. As output, the server creates an adjusted version of each recipe with updated ingredient quantities and, if necessary, replacement ingredients that conform to disallowed ingredient and allergen constraints.

### Step 14

The server optionally refines adjusted recipes using an additional prompt sentence to the generative AI model. The server takes, as input, an original recipe text and the desired adjustment (for example “reduce calories by 20 percent while keeping protein high and avoiding eggs and peanuts”). The server constructs a refinement prompt sentence that embeds both the original recipe and the adjustment conditions. The server transmits this refinement prompt to the generative AI model and receives adjusted recipe text. As output, the server obtains an updated recipe description that reflects the requested nutritional modification and parses it again into structured recipe data using the same parsing and normalization procedures.

### Step 15

The server re-computes nutrient amounts for the adjusted recipes and validates the updated nutrient-intake balance. The server takes, as input, the adjusted structured recipe data, re-queries the nutrient-information storage apparatus for each ingredient, and recomputes nutrient vectors using the updated quantities. The server compares the recomputed nutrient amounts with nutrient-intake criteria, as in previous comparison steps, to ensure that the nutrient-intake balance now falls within target ranges. As output, the server produces a finalized set of recipes with confirmed nutrient profiles.

### Step 16

The server aggregates ingredient requirements for the entire planning period. The server takes, as input, all finalized recipes and their normalized ingredient lists, and groups ingredient entries by canonical ingredient identifier. The server sums quantities across all recipes for each ingredient, converting units when necessary to maintain a consistent base unit. As output, the server generates an aggregated ingredient requirement list that specifies total required quantities for each ingredient over the predetermined period.

### Step 17

The server converts aggregated ingredient requirements into merchandise information suitable for an electronic transaction apparatus. The server takes, as input, the aggregated ingredient list with total quantities and uses mapping tables to associate canonical ingredient identifiers with generic merchandise descriptors and typical package sizes. The server computes the number of packages required by dividing total quantities by package size and rounding as needed. As output, the server creates merchandise entries that include product-category codes, package counts, and any other attributes required by the electronic transaction apparatus.

### Step 18

The server formats purchase-candidate data according to an interface specification of the electronic transaction apparatus. The server takes, as input, the merchandise entries and quantity information, and arranges them into a data structure specified by the external commerce API, such as a list of line items with identifiers and counts. The server assigns each line item to a designated shopping-cart or order-draft identifier. As output, the server produces purchase-candidate data that can be directly consumed by the electronic transaction apparatus to create a purchase-candidate state.

### Step 19

The server transmits the purchase-candidate data to the electronic transaction apparatus and receives a purchase-candidate state identifier. The server takes, as input, the formatted purchase-candidate data and sends it via an API call to the commerce system. The commerce system creates or updates a shopping cart or pre-order and returns an identifier or URL representing the purchase-candidate state. As output, the server stores this identifier in association with the current meal plan and prepares it for inclusion in the response to the terminal.

### Step 20

The server composes display data for the terminal that includes recipe, nutrition, and purchase information. The server takes, as input, the finalized structured recipe data, the computed nutrient amounts, and the purchase-candidate state identifier. The server organizes this information into a presentation-oriented structure, such as grouping recipes by day and attaching nutrient summaries and purchasing links. As output, the server generates display data that can be rendered by the terminal to show the weekly meal plan, nutritional breakdowns, and a control to access the prepared purchase-candidate state.

### Step 21

The terminal receives the display data from the server and renders user interface elements. The terminal takes, as input, the display data and uses client-side logic to generate visual components such as recipe lists, ingredient tables, nutrient graphs, and a “Purchase Ingredients” button. The terminal converts numeric and textual data into formatted text, charts, and interactive controls. As output, the terminal presents a complete view of the meal plan and purchasing options to the user on the display.

### Step 22

The user reviews the proposed meal plan and purchasing information using the terminal. The user reads recipe titles, ingredients, and instructions, and examines nutritional summaries. Based on this input, the user decides whether to accept the plan, request regeneration by changing inputs, or proceed to purchase. As output, the user generates a new action, such as selecting the “Purchase Ingredients” control.

### Step 23

The terminal responds to the user's purchase action by opening the purchase-candidate state on the electronic transaction apparatus. The terminal takes, as input, the purchase-candidate state identifier or URL included in the display data, and initiates a navigation or application call to the commerce system. The terminal loads the shopping-cart or pre-order page from the electronic transaction apparatus. As output, the terminal displays the order-ready list of items and quantities to the user.

### Step 24

The user uses the terminal to inspect and optionally modify the items in the purchase-candidate state on the electronic transaction apparatus. The user may change quantities, remove items, or add further items. As input, the commerce interface receives the user's cart edits. As output, the commerce system updates the purchase-candidate state and, upon final confirmation, processes the order and initiates real-world fulfillment of the ingredients.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional meal-recommendation and recipe-generation systems typically rely on static rule sets or simple filtering logic executed by generic application servers. Such systems merely match user-specified constraints (for example, budget, disliked ingredients, or allergens) against a predefined recipe database and then present matching items. As a result, these systems exhibit several technical limitations.

First, existing systems do not tightly integrate heterogeneous user-related data streams—such as constraint information, health-related information, and emotion-related information—into a unified, machine-interpretable context that can be consumed by a generative AI model. In many deployments, server-side components treat these data sets independently, or use them only as coarse post-filters. This leads to inefficient use of processing resources, because large numbers of unsuitable candidates must be generated and then discarded, and also results in sub-optimal personalization.

Second, typical architectures do not provide a structured, programmatic mechanism on the server to synthesize a rich prompt sentence that encodes detailed user context, health metrics, and emotional state in a form optimized for a generative AI model. Instead, prompts are often manually crafted, static, or loosely coupled to back-end data structures. This decoupling degrades the quality and consistency of generated outputs, causes unnecessary network round-trips and retries between the server and the AI backend, and increases overall latency.

Third, conventional systems treat e-commerce integration as an independent process that is manually operated by the user after a recipe is presented. Ingredient extraction from generated text, mapping to purchasable items, and preparation of a cart state on an electronic commerce platform are frequently performed on the client side or through ad-hoc scripts. This fragmented workflow leads to redundant parsing on multiple tiers, inconsistent item mapping, and increased server and network load, as well as higher error rates when users attempt to purchase ingredients.

Fourth, most systems that consider user preferences or health data employ batch-style analytics that are not looped back into the real-time recipe-generation pipeline. Model training and preference estimation, if present, are not integrated with prompt generation and ranking of the generative AI model's outputs at the server. Consequently, the server cannot adaptively optimize its prompts or selection logic based on newly acquired feedback, and cannot reduce the search space before invoking the generative AI model. This results in inefficient compute usage on the AI backend and slower response times.

Fifth, conventional architectures rarely incorporate real-time emotion estimation from multimodal inputs (audio and image) at the server level in a way that systematically influences both (i) the content generated by a generative AI model and (ii) the presentation mode of that content. Existing emotion-aware interfaces, if any, are usually implemented solely in the client application and do not feed back into the server-side control of prompt formation, result ranking, and message generation. This separation prevents the server from performing coordinated, stateful optimization of resource usage and user interaction flow based on the user's emotional state.

Accordingly, there is a need for an improved computer-implemented system and server architecture that: (i) unifies constraint information, health-related information, and emotion-related information into a single, machine-interpretable user context; (ii) programmatically generates optimized prompt sentences for a generative AI model based on this context; (iii) parses and normalizes AI-generated outputs on the server, including robust extraction and mapping of ingredient information to items on an electronic commerce platform; (iv) continuously learns from user feedback and health-related data via a machine-learning model to update internal preference and health parameters; and (v) uses estimated emotional states to dynamically adjust both the prompt content and the presentation of generated meal proposals. By addressing these technical issues at the server level, the invention improves the functioning of the computer system as a whole, reducing redundant computation and network traffic, lowering latency, and increasing robustness and accuracy of the end-to-end meal-planning and purchasing workflow.

The specific processing by the specific processing unit 290 of the data processing device 12 in Application Example 2 is realized by the following means.

The present invention provides a server comprising a processor configured to acquire constraint information related to meals, including budget information, ingredient-avoidance information, and allergen-avoidance information, from a terminal device operated by a user; to further acquire user information including the constraint information, health-related information comprising at least one of physical-activity information and nutritional-status information, and emotion-related information indicating an emotional state of the user; to analyze the user information and, based on a result of the analysis, generate a prompt sentence that instructs a generative AI model to generate a meal proposal for a predetermined period including a plurality of meal contents satisfying the constraint information and the health-related information and having characteristics corresponding to the emotion-related information; to input the prompt sentence to the generative AI model and obtain, from the generative AI model, a generation result including a plurality of cooking procedures constituting the meal proposal and ingredient information corresponding to the cooking procedures; to extract the ingredient information from the generation result, specify required articles based on the extracted ingredient information and item-identification information associated with merchandise-information records, and register the specified articles, via an electronic commerce platform, as purchase-processing information in a state immediately before order placement; to receive evaluation information from the user regarding dishes included in the meal proposal together with the health-related information, and to input the evaluation information and the health-related information as learning data to a machine-learning model so as to update parameters representing user preferences and health states; and to optimize at least one of a subsequent prompt sentence to be input to the generative AI model and a composition of dishes selected from subsequent generation results based on the updated parameters, while also changing at least one of a content of the prompt sentence, a presentation order of the meal proposal, and wording used in presentation of the meal proposal in accordance with an estimated emotional state obtained from acoustic information and image information by at least one of speech-recognition processing and image-recognition processing. This enables the server to internally synthesize heterogeneous user-related data into a unified context, to generate and refine prompt sentences for the generative AI model that are optimized for both computational efficiency and personalization, to centrally perform robust parsing and normalization of AI-generated outputs including consistent mapping to purchasable items on an electronic commerce platform, and to adapt its real-time generation and presentation behavior based on continuously updated preference, health, and emotion parameters, thereby improving overall system performance, reducing redundant processing and network traffic, and enhancing the reliability and responsiveness of computer-implemented meal-planning and purchasing operations.

The term “constraint information” refers to information indicating one or more limitations or conditions specified by a user with respect to meals, including at least budget limitations, ingredient-avoidance conditions, and allergen-avoidance conditions.

The term “budget information” refers to numerical or categorical information indicating a range, limit, or target amount of monetary expenditure that a user permits for purchasing ingredients or meals over a specified period.

The term “ingredient-avoidance information” refers to information indicating one or more food components or ingredient categories that a user wishes to exclude from meals, for reasons such as dislike, religious restrictions, or dietary preferences.

The term “allergen-avoidance information” refers to information indicating one or more substances or ingredient types that must be excluded from meals in order to avoid an allergic reaction in the user.

The term “user information” refers to aggregated information associated with a user, including constraint information, health-related information, and emotion-related information, that is used by the system to generate and present meal proposals.

The term “health-related information” refers to information representing a physical condition of a user, including at least one of physical-activity information and nutritional-status information, and optionally other biometric or medical indicators.

The term “physical-activity information” refers to information indicating a user's amount or pattern of body movement or exercise, such as step counts, energy expenditure, workout duration, or similar activity metrics obtained from a sensing device or application.

The term “nutritional-status information” refers to information indicating a user's nutritional state, such as calorie intake, macronutrient intake, body weight, body mass index, or other indicators of diet-related health.

The term “emotion-related information” refers to information indicating or derived from an emotional state of a user, obtained from sources such as acoustic features, facial expressions, textual input, or interaction patterns.

The term “emotional state” refers to a psychological condition of a user at a given time, such as stress, happiness, sadness, or calmness, inferred or estimated from emotion-related information.

The term “generative AI model” refers to a machine-implemented computational model configured to generate new data, including natural-language text representing meal proposals, in response to a prompt sentence, based on learned parameters obtained from training on existing data.

The term “prompt sentence” refers to a structured natural-language or machine-interpretable instruction string generated by the processor and provided as input to the generative AI model to request generation of a meal proposal under specified conditions.

The term “meal proposal” refers to a set of one or more meal contents, such as dishes or menus, for a predetermined period, generated by the generative AI model or derived therefrom, and intended to satisfy user-specific constraints and health-related conditions.

The term “meal content” refers to information representing at least one dish or combination of dishes for a given eating occasion, including identifiers such as a dish name and associated preparation details.

The term “cooking procedure” refers to a sequence of instructions or steps that describe how to prepare a dish or meal content using specified ingredients.

The term “ingredient information” refers to data indicating types, names, quantities, and optionally units or attributes of food components to be used in a cooking procedure.

The term “generation result” refers to output data produced by the generative AI model in response to a prompt sentence, including at least a plurality of cooking procedures and associated ingredient information.

The term “article” refers to a purchasable item, such as a packaged food product or ingredient unit, that is represented in merchandise-information records and may be ordered through an electronic commerce platform.

The term “item-identification information” refers to information that uniquely or distinctively identifies an article in a merchandise-information record, such as a code, stock-keeping unit, or catalog identifier.

The term “merchandise-information record” refers to stored data describing a purchasable article, including at least item-identification information and one or more attributes such as name, size, and price.

The term “electronic commerce platform” refers to a computer-implemented transactional system that enables remote purchase of articles via a communication network, including functionality to manage shopping carts, orders, and payment processing.

The term “purchase-processing information” refers to data used by an electronic commerce platform to process a prospective transaction, including at least item identifiers, quantities, and optionally pricing and delivery parameters.

The term “state immediately before order placement” refers to a configuration of purchase-processing information in which required articles have been registered in association with a user account or session on an electronic commerce platform, such that an order can be placed by executing a final confirmation operation.

The term “terminal device” refers to a user-operated computing apparatus, such as a mobile device or personal computer, configured to communicate with the server, provide input interfaces, and present meal proposals and related information.

The term “presentation information” refers to data generated by the processor for transmission to a terminal device, defining content and formatting for display or output of meal proposals, such as dish lists, instructions, and messages.

The term “presentation mode” refers to one or more aspects of how presentation information is output to the user, including order, wording, layout, style, or emphasis, which may be varied according to the user's emotional state.

The term “evaluation information” refers to information provided by a user that assesses or rates dishes or meal proposals, including but not limited to numerical ratings and textual comments.

The term “machine-learning model” refers to a computational model, distinct from the generative AI model described above, that is trained on learning data to infer or update parameters representing user preferences or health states.

The term “parameters representing user preferences and health states” refers to internal values or vectors maintained by the system that quantitatively encode a user's likes, dislikes, and health-related conditions for use in optimization of prompt generation and dish selection.

The term “learning data” refers to data sets provided to a machine-learning model for training or updating, including evaluation information, health-related information, and optionally other contextual information.

The term “optimize” refers to the process of adjusting or selecting at least one of a prompt sentence or a composition of dishes so as to improve alignment with user preferences, health conditions, or system performance criteria according to a defined objective function.

The term “composition of dishes” refers to the set, selection, or arrangement of one or more dishes included in a meal proposal output or selected from the generation result.

The term “emotion-estimation input information” refers to data used to estimate the emotional state of a user, including at least acoustic information and image information acquired from user interactions.

The term “acoustic information” refers to digital data representing sound captured from the user, including speech signals, used for at least one of speech recognition and emotion estimation.

The term “image information” refers to digital image or video data representing at least a portion of a user, such as a face, used for at least one of image recognition and emotion estimation.

The term “speech-recognition processing” refers to processing that analyzes acoustic information to obtain textual content or features usable for recognizing speech and estimating an emotional state.

The term “image-recognition processing” refers to processing that analyzes image information to detect or classify visual features, such as facial expressions, usable for estimating an emotional state.

The term “presentation order” refers to the sequence in which meal proposals or dishes are displayed or otherwise presented to the user.

The term “wording used in presentation” refers to textual expressions included in presentation information, such as descriptions or messages attached to meal proposals, which may be varied according to system state or user emotion.

In the following embodiments, a server, a terminal, and a user cooperate to implement the claimed system. The embodiments described below are illustrative examples that support the scope of the claims and can be implemented in various hardware and software configurations.

### A. System Configuration

A server includes at least one processor, a main memory, a non-volatile storage device, a network interface, and an interface to at least one electronic commerce platform. The server is implemented, for example, as a rack-mounted computer or a cloud-based virtual machine. The server executes a server-side program on an operating system. The server-side program is implemented, for example, using a web framework such as a Python-based framework, and communicates with external services via HTTP over a network.

A terminal includes a processor, a display, an input device such as a touch panel, a microphone, and a camera, and communicates with the server via a wireless or wired network. The terminal executes an application, for example a native application implemented with a cross-platform user interface framework, or a web browser rendering a web application.

A user operates the terminal to input constraint information, health-related information, and feedback regarding meal proposals, and to confirm purchase of ingredients via the electronic commerce platform.

The server is coupled to at least one data storage device storing:
user profile records, including identifiers, demographic attributes, and authentication data;
constraint information records, including budget information, ingredient-avoidance information, and allergen-avoidance information;
health-related records, including physical-activity information and nutritional-status information;
emotion-related records, including emotion-estimation results derived from acoustic and image information;
recipe and meal-plan records, including generated cooking procedures and ingredient information;
feedback records, including evaluation information from the user;
merchandise-information records, including item-identification information and mapping from ingredient information to purchasable articles.

The server further accesses an external generative AI model via a network, and accesses at least one external emotion-analysis service and at least one electronic commerce platform via application programming interfaces (APIs).

### B. Acquisition and Representation of User Information

The terminal presents to the user an input interface for constraint information. The interface includes controls for numeric budget values, checklists for ingredients to avoid, and checklists or text fields for allergens. The terminal collects this information and structures it into a constraint information data structure, for example as key-value pairs including a budget field, an ingredient-avoidance field, and an allergen-avoidance field.

The server receives the constraint information from the terminal and normalizes each field. The server converts the budget field into a normalized numeric representation, for example an integer representing a currency amount for a given period. The server maps free-text ingredient names and allergen names to canonical ingredient identifiers using a lookup table stored in the merchandise-information records. By performing this mapping once at the server, the system reduces duplication of normalization logic in the terminal and achieves consistent identification of ingredients across multiple modules.

The server retrieves health-related information for the user from external health services. The server receives physical-activity information such as daily step counts, workout durations, and estimated energy expenditure, and nutritional-status information such as calorie intake and body-weight measurements. The server stores these data values in a time-series structure indexed by timestamps and by user identifiers.

The terminal optionally captures acoustic information from the microphone and image information from the camera while the user interacts with the application. The terminal transmits the captured audio and image data to the server, together with metadata indicating the capture time and context. The server stores these data temporarily for emotion estimation.

The server maintains a unified user information structure including references to the constraint information, the health-related information, and the emotion-related information. The unified structure is implemented, for example, as a record containing fields for constraint vectors, health feature vectors, and emotion labels, thereby enabling efficient computation of derived features and reuse across multiple modules.

### C. Emotion Estimation and Representation

The server analyzes the acoustic information and image information to estimate an emotional state of the user. The server performs speech-recognition processing on the acoustic information to obtain textual content, and also extracts prosodic features such as pitch, energy, and speaking rate. The server applies a trained classification model, such as a recurrent neural network or transformer-based model, to the combination of textual features and prosodic features to estimate probabilities of emotion categories such as “stressed,” “calm,” “happy,” or “sad.”

In parallel, the server performs image-recognition processing on the image information. The server detects a face region using a convolutional-neural-network-based detector and extracts facial features such as mouth curvature, eyebrow position, and eye openness. The server feeds these visual features into a trained classifier, for example a convolutional neural network followed by fully connected layers, to obtain probabilities of emotion categories.

The server fuses the results of acoustic-based and image-based classifiers by computing a weighted average or by feeding the probability vectors into a small feed-forward neural network. The server selects the emotion category having the highest fused probability and stores this category, together with confidence values, as the emotion-related information in the user information structure. This fusion process reduces noise from each modality and improves classification accuracy compared with using either modality alone, thereby improving the robustness of downstream prompt generation and presentation control.

### D. Generation of Prompt Sentences for the Generative AI Model

The server constructs a prompt sentence for the generative AI model based on the user information. The server first derives feature values capturing the user's current context. These features include normalized budget, a vector indicating forbidden ingredients and allergens, summary statistics of recent physical-activity and nutritional-status values, and the current emotional state.

The server converts these feature values into human-readable phrases using templates stored in a configuration repository. For example, when the budget is a weekly amount, the server generates a phrase such as “The user's weekly budget is 5000 units of currency.” When the user's calorie target is 2000 kilocalories per day with a preference for high protein, the server generates a phrase such as “The user's daily calorie target is 2000 kcal with high protein and low sugar.” When the user is detected as stressed, the server generates a phrase such as “The user is currently stressed and prefers simple, comforting dishes.”

The server concatenates these phrases with explicit instructions specifying the desired output structure of the generative AI model. The server, for example, constructs a prompt sentence such as:

“The user's daily calorie target is 2000 kcal with high protein and low sugar. The weekly budget is 5000 units of currency. The user dislikes eggplant and mushrooms and must avoid wheat. The user is currently stressed and prefers simple, comforting dishes. The user has rated spicy dishes poorly in the past and rated pasta dishes highly. Using the generative AI model, please generate a 7-day dinner plan. Include at least two pasta-based dinners using gluten-free pasta. For each day, output: a dish name, a list of ingredients with quantity and unit, an approximate calorie value, macronutrient amounts for protein, fat, and carbohydrates, and step-by-step cooking instructions. Exclude eggplant, mushrooms, wheat, and very spicy ingredients.”

The server logs the prompt sentence and associates it with the corresponding user information structure. By encoding detailed constraints and context into a single prompt sentence, the server reduces the need for multiple iterations with the generative AI model and decreases the amount of post-processing to remove unsuitable candidates, resulting in lower network load and processing latency.

### E. Generative AI Model Structure and Usage

In one embodiment, the generative AI model is a transformer-based neural network pre-trained on large corpora of text and fine-tuned on recipe and nutrition datasets. The model includes multiple layers of self-attention and feed-forward sublayers, and maintains parameter matrices representing token embeddings, positional encodings, and layer weights. The model generates text sequences token by token conditioned on the prompt sentence.

The server communicates with the generative AI model via an API. The server sends the prompt sentence and configuration parameters such as maximum sequence length, sampling temperature, and top-k or top-p sampling thresholds. The model computes, for each decoding step, a probability distribution over vocabulary tokens using the softmax function applied to the output of the final transformer layer.

The server can, in some embodiments, host a generative AI model locally. In that case, the server stores model weights in memory, and the processor performs matrix multiplications and non-linear activation functions according to the transformer architecture. The server may use dedicated linear-algebra libraries or hardware acceleration units to speed up matrix operations.

The server receives the output text representing the generation result. The generation result includes human-readable descriptions of dishes, ingredients, quantities, and cooking steps. The server verifies that the generation result follows the requested structure and, if deviations are detected, constructs a corrective prompt sentence requesting the generative AI model to reformat or correct specific portions. This interaction is performed in a controlled manner so that the server avoids large numbers of unstructured responses.

### F. Parsing and Normalization of Generation Results

The server parses the generation result to extract structured ingredient information and cooking procedures. The server segments the text into sections corresponding to each day and each dish. For each section, the server identifies lines or phrases that represent ingredient entries and lines that represent steps.

The server uses rule-based patterns and tokenization to extract ingredient names, quantities, and units. The server maps extracted ingredient names to canonical identifiers using the same merchandise-information table used for constraint normalization. The server converts units into standardized metric units using a conversion table. By maintaining centralized conversion tables and mapping logic, the server prevents inconsistent interpretations of units and ingredient names, which could otherwise arise if similar processing were performed independently at multiple tiers.

The server verifies that none of the ingredients belong to the forbidden categories specified by the constraint information. When a violation is detected, the server either discards the dish or constructs a secondary prompt sentence requesting the generative AI model to replace only the violating ingredients while maintaining other structure. This targeted replacement avoids regenerating entire meal plans and thus reduces unnecessary computation and network traffic.

### G. Mapping Ingredients to Articles and Preparing E-Commerce Transactions

The server uses the normalized ingredient information to identify purchasable articles in the merchandise-information records. The server maintains a mapping from canonical ingredient identifiers to one or more item-identification information values. In some embodiments, the mapping includes rules for selecting package sizes or brands based on quantity requirements and user preferences.

The server aggregates ingredient requirements across all dishes in a meal proposal. The server sums required quantities per ingredient and selects appropriate articles such that package sizes and counts cover the total quantity with minimal surplus. The server uses a simple optimization algorithm, such as a greedy algorithm or integer linear programming, to minimize cost or waste. This optimization algorithm relies on the structured data representation created by the earlier normalization and significantly reduces the complexity of constructing a purchase order compared with manual or client-side methods.

The server then communicates with the electronic commerce platform via its API. The server transmits lists of item-identification information and quantities and receives a response indicating cart identifiers, price breakdowns, and stock availability. The server stores these data and associates them with the user information and the meal proposal. The cart is placed in a state immediately before order placement, in which the user can complete the transaction by performing a minimal confirmation operation on the terminal.

By performing the mapping and optimization centrally at the server, the system avoids repeated search and parsing operations that would otherwise be performed each time the user adjusts a recipe or reopens the application. The server can reuse cached mapping and optimization results, thereby reducing load on the electronic commerce platform and network bandwidth.

### H. Learning User Preferences and Health-Aware Optimization

The server maintains a separate machine-learning model to predict user preferences and to estimate health-related compliance of dishes. The preference model is, for example, an embedding-based neural network that takes as input a representation of a dish and a representation of a user. The dish representation includes binary indicators or counts of ingredients, cuisine type, cooking time, and previous usage frequencies. The user representation includes demographic information, past ratings, and aggregated health-related features.

The server trains the machine-learning model using evaluation information and health-related information as learning data. The server constructs training examples in which the input is a pair consisting of a dish feature vector and a user feature vector, and the target is a numerical rating or a binary indicator of acceptance. The server defines a loss function, such as mean squared error or cross-entropy, and adjusts weights of the model using gradient-descent optimization. The server may use techniques such as mini-batch training, regularization, and data augmentation (for example, slight perturbation of ingredient features) to improve generalization.

The server periodically retrains or fine-tunes the model with new feedback data. When a user provides evaluation information for a dish, the server records the rating, textual comments, and context. The server uses this information to update the model and to refine the internal parameters representing the user's preferences and health states.

The server uses the trained model to score dishes generated by the generative AI model. The server computes a preference score representing an estimated rating and a health score representing alignment with nutritional targets derived from health-related information. The server combines these scores according to a weighted formula and selects dishes or meal compositions that maximize the combined score while satisfying constraint information.

By integrating this scoring and optimization step in the server, the system reduces the need to rely solely on the generative AI model to select suitable dishes. The server can discard low-scoring candidates before presenting them to the user, thereby decreasing cognitive load on the user and reducing the number of interactions required to reach an acceptable meal plan.

### I. Emotion-Aware Prompt and Presentation Control

The server uses the estimated emotional state not only as a descriptive attribute but as a control parameter that influences both the content of the prompt sentence and the presentation mode on the terminal. When the user is stressed, the server modifies the prompt sentence to favor simpler recipes and comforting dishes. For example, the server may add phrases such as “The user is feeling stressed and wants a simple, relaxing dinner. Please avoid complicated cooking procedures and limit preparation time to 30 minutes.”

The server also generates emotion-adapted textual messages to accompany recipes. The server may send a short auxiliary prompt sentence to the generative AI model, such as:

“Given that the user is stressed, generate a short, encouraging introduction (one sentence) for the following recipe: grilled chicken with vegetable soup.”

The server then attaches the generated introduction to the recipe record and includes it in the presentation information sent to the terminal.

The terminal adjusts display elements such as ordering of dishes, emphasis, and message wording based on the presentation information. For instance, the terminal may highlight particularly suitable recipes for a stressed user and display encouraging messages near them. This coordinated use of emotion-related information across prompt generation and presentation control provides a consistent, stateful user experience and reduces unnecessary browsing and decision time.

### J. Technical Effects and Improvement of Computer Technology

The described architecture provides several technical advantages beyond mere automation of human decision making:
1. The server unifies disparate data sources (constraint information, health-related information, emotion-related information, and feedback) into a structured user information representation. This unified representation enables efficient feature computation and reuse across prompt generation, scoring, and e-commerce mapping modules, reducing repeated conversions and lookups.
2. The server generates optimized prompt sentences that encode rich context and explicit constraints for the generative AI model. By doing so, the server reduces the number of iterations needed to obtain satisfactory generation results and minimizes the size of the search space. This leads to fewer remote calls to the generative AI model, reduced network traffic, and lower latency.
3. The server performs centralized parsing and normalization of generation results, including unit conversion and mapping to canonical ingredient identifiers. This reduces inconsistency and error rates relative to performing similar operations independently on multiple clients, and improves robustness when new ingredients or formats appear in the generated text.
4. The server integrates a machine-learning model that continuously learns from evaluation information and health-related information, and feeds updated parameters back into both prompt generation and selection logic. This feedback loop allows the server to pre-filter and re-rank candidate dishes before presenting them, reducing computational load on both the generative AI model and the electronic commerce platform.
5. The server performs multimodal emotion estimation and uses the estimated emotional state to dynamically control both the prompt sentence content and the presentation mode. This stateful, context-aware control is implemented through explicit algorithms and data structures, rather than being left to client-side heuristics, and leads to more efficient user interactions and reduced average time to finalize a meal plan and associated purchase.
6. The server employs specific neural-network architectures (transformer-based generative AI model for text generation and classification models for preference prediction and emotion estimation), specific loss functions, and parameter-update methods. The server's integration of these models into a coordinated dataflow, with clearly defined feature representations and control signals, improves overall computational efficiency relative to naive combinations of independent services.

In sum, the described system improves the functioning of the computer network that implements it by reducing redundant computations, lowering communication overhead, increasing robustness of data normalization and mapping, and enabling more accurate and faster generation of meal proposals and preparation of e-commerce transactions. These improvements stem from specific technical mechanisms in the server's data structures, model architectures, and control logic, and are not merely a computerization of a human workflow.

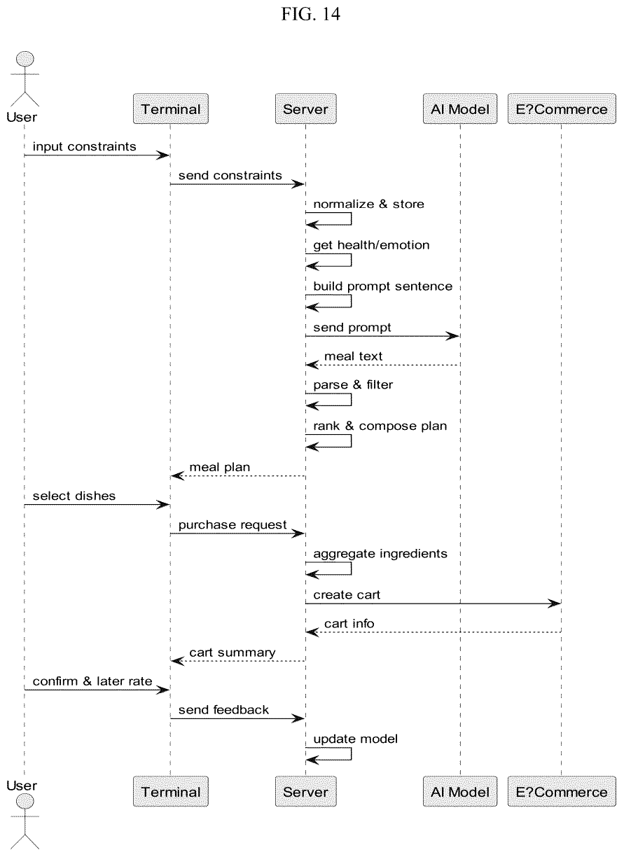

The following describes the processing flow using FIG. 14.

### Step 1

User operates the terminal to input constraint information.

User views, on the terminal display, an input screen that includes fields for budget, disliked ingredients, and allergens to avoid. User types numeric values for budget, selects disliked ingredients from a list, and selects allergens from another list or enters them as free text.

Input: raw user keystrokes and touch events.

Terminal converts the user actions into structured data by binding each UI control to a field name (for example “budget”, “disliked_ingredients”, “allergens”) and assembling these into a data object. Terminal performs basic validation, such as checking that the budget is a positive number and that at least one field is filled.

Output: a validated constraint information object held in the memory of the terminal.

### Step 2

Terminal sends constraint information to the server.

Terminal serializes the constraint information object into a message (for example JSON text) and attaches user identification and authentication data. Terminal sends this message to a predefined API endpoint on the server using a secure communication protocol.

Input: validated constraint information object and user identification data.

Terminal performs data formatting (serialization) and encapsulates the serialized data into a network packet. Terminal uses a networking stack to transmit the packet through a communication interface.

Output: an HTTP request containing the constraint information, delivered to the server.

### Step 3

Server receives and normalizes constraint information.

Server accepts the HTTP request at its network interface and passes the body to an application component. Server parses the serialized message and reconstructs the constraint information object in server memory.

Input: serialized constraint information in the request body.

Server converts the budget string into a numeric value, converts ingredient names to lowercase and trims whitespace, and tokenizes allergen names. Server looks up each ingredient and allergen token in an internal mapping table and replaces text labels with canonical identifiers.

Output: a normalized constraint information record stored in a database and linked to the user.

### Step 4

Server acquires and aggregates health-related information.

Server determines whether the user has allowed access to external health data sources. If allowed, server calls external health-service APIs using stored access tokens.

Input: user identifier and access tokens, plus API responses containing raw step counts, energy expenditure, calorie intake, and body weight measurements.

Server converts timestamps from external services into a unified time zone and aligns measurements by day. Server computes daily totals and averages, such as total steps per day and average calorie intake. Server then writes these aggregated values into health-related records in the database.

Output: a time-series health-related data set indexed by user and date.

### Step 5

Terminal captures emotion-estimation input and sends it to the server.

Terminal records audio using the microphone while the user speaks and captures an image or short video using the camera while the user views the application.

Input: continuous analog sound and optical signals.

Terminal digitizes the signals, encoding the audio into an audio file and compressing the image into an image file. Terminal associates timestamps and user identifiers with these files and encapsulates them in a message. Terminal transmits the message to the server via a secure API.

Output: a request to the server that includes acoustic information and image information for emotion estimation.

### Step 6

Server estimates an emotional state from acoustic and image information.

Server receives the audio and image data and forwards them to internal or external analysis modules.

Input: acoustic information and image information associated with the user.

Server performs feature extraction on the acoustic information, such as computing mel-frequency coefficients, pitch contours, and energy statistics. Server performs feature extraction on the image information, such as detecting a face region and computing facial landmarks. Server then applies trained classification models to the acoustic features and image features to obtain probability distributions over emotion categories. Server combines these probability distributions, for example by weighted averaging, and selects the category with the highest combined probability as the current emotional state.

Output: an emotion-related record containing an emotional state label and confidence values, stored in association with the user.

### Step 7

Server constructs a unified user information structure.

Server collects, from storage, the normalized constraint information, the aggregated health-related information, and the latest emotion-related information for the user.

Input: constraint record, health-related records, emotion-related record.

Server converts these records into vector or structured fields, such as a numeric budget value, a binary vector marking forbidden ingredients, a vector of health indicators (for example average daily calorie intake and physical-activity level), and an encoded emotional state. Server then stores these fields in a composite data structure in memory that will be passed to subsequent modules.

Output: a unified user information structure representing all relevant parameters for prompt generation.

### Step 8

Server generates a prompt sentence for the generative AI model.

Server analyzes the unified user information structure and uses template rules to generate language fragments.

Input: unified user information structure containing constraint, health, and emotion data.

Server converts numeric values and categorical values into descriptive phrases; for example, it maps a budget value to a phrase about weekly spending, and maps health indicators to phrases about calorie targets and macronutrient preferences. Server then assembles these phrases into a single coherent prompt sentence and appends explicit instructions that define required output fields and prohibited items.

Output: a complete prompt sentence in text form suitable as input to the generative AI model.

### Step 9

Server sends the prompt sentence to the generative AI model and receives a generation result.

Server calls an inference interface exposed by the generative AI model.

Input: prompt sentence text and generation parameters such as maximum length and sampling settings.

Server transmits the prompt sentence and parameters to the generative AI model and waits for a response. The generative AI model performs internal matrix operations on token embeddings and attention layers to generate a sequence of output tokens. Server reads the output as a contiguous text string and associates it with the originating user and prompt sentence.

Output: a generation result text that contains candidate meal proposals, ingredients, and cooking procedures.

### Step 10

Server parses and structures the generation result.

Server interprets the generation result text and splits it into sections representing different days and dishes.

Input: raw generation result text.

Server searches for patterns such as “Day 1”, “Ingredients:”, “Steps:” and similar markers. Server extracts lines belonging to ingredient lists and identifies quantities and units through tokenization and pattern matching. Server standardizes unit names and converts units into a chosen metric system using a conversion table. Server also separates cooking procedures into ordered steps.

Output: a structured representation of candidate dishes, each including a normalized ingredient list and a sequence of cooking steps.

### Step 11

Server filters dishes against constraint information and regenerates if necessary.

Server compares each ingredient identifier in the structured dishes with the constraint information.

Input: structured dishes and normalized constraint record.

Server checks each ingredient against the lists of ingredients to avoid and allergens to avoid. If a dish includes a forbidden item, server flags that dish as invalid. If too many dishes are invalid, server constructs an additional prompt sentence specifying that certain ingredients must not appear and sends this modified prompt sentence back to the generative AI model.

Output: a filtered set of dishes that satisfy constraint information or a revised generation result after regeneration.

### Step 12

Server evaluates and ranks dishes using a preference model.

Server loads a trained preference model and associated parameters from storage.

Input: filtered dishes and user-specific feature vectors derived from past feedback and health-related information.

Server converts each dish into a feature vector, encoding ingredient identifiers, cooking time, and nutritional profile. Server concatenates or otherwise combines this vector with a user feature vector that encodes preference and health parameters. Server feeds the combined vector into the preference model to obtain a numerical score for each dish. Server sorts dishes in descending order of scores.

Output: a ranked list of dishes with associated preference scores and health scores.

### Step 13

Server composes a meal proposal for a predetermined period.

Server selects top-ranked dishes from the list, while enforcing diversity constraints such as limiting repetition of similar main ingredients.

Input: ranked dish list and selection constraints.

Server iteratively selects dishes for each day of the period, checking for conflicts such as repeated main ingredients or excessive calorie totals for a day. If a dish violates a diversity or health constraint, server selects the next highest-ranked dish. The selection process continues until the predetermined number of days is filled.

Output: a meal proposal object containing a schedule of dishes and associated ingredient lists and cooking steps.

### Step 14

Server generates presentation information based on the meal proposal and emotional state.

Server retrieves the current emotional state for the user and uses it to adjust the wording and ordering of dishes.

Input: meal proposal object and emotion-related record.

Server generates short textual messages for each dish, possibly by sending small auxiliary prompt sentences to the generative AI model and embedding the dish name and emotional state into the prompt. Server also determines the order of dishes to be presented, for example highlighting simpler or more comforting dishes first when the user is stressed. Server packs dish details, nutritional summaries, and emotional messages into a presentation data structure.

Output: presentation information ready to be transmitted to the terminal.

### Step 15

Terminal receives and displays the meal proposal.

Terminal receives the presentation information from the server as a response to an API call.

Input: presentation data structure including scheduled dishes, ingredients, steps, and messages.

Terminal parses the structure and renders a user interface that shows the overall plan and allows the user to tap a particular day or dish for more detail. Terminal lays out text fields, icons, and buttons according to the presentation mode specified in the data (for example, emphasizing certain messages when the user is stressed).

Output: visual and optional audio output to the user representing the meal proposal and associated information.

### Step 16

User reviews the meal proposal and selects dishes or days for ingredient purchase.

User examines the list of proposed dishes and, via the terminal, selects which days or dishes to include in an ingredient purchase.

Input: rendered UI elements and user actions such as taps on selection controls.

Terminal maps the user's selection to identifiers of dishes in the meal proposal. Terminal then assembles a purchase request object that lists the selected dishes and sends this object to the server for ingredient mapping and cart preparation.

Output: a purchase request delivered from the terminal to the server.

### Step 17

Server aggregates ingredients and maps them to articles.

Server receives the purchase request and retrieves corresponding dish definitions.

Input: purchase request and structured dishes from the meal proposal.

Server combines ingredient lists from all selected dishes, grouping entries by canonical ingredient identifier and summing required quantities. Server compares the summarized requirements with the merchandise-information records and selects appropriate articles to cover the required quantities with minimal waste and cost. Server construct a list of item-identification information values and associated quantities.

Output: an aggregated item list suitable for submission to an electronic commerce platform.

### Step 18

Server prepares a shopping cart in an electronic commerce platform.

Server connects to the electronic commerce platform API and transmits the aggregated item list.

Input: aggregated item list and user account information for the platform.

Server sends structured requests adding each item to a virtual shopping cart. Server receives confirmation data including cart identifier, availability, and pricing. Server stores this information in a cart record associated with the user and the current meal proposal.

Output: a cart state in the platform in a condition immediately before order placement, and cart summary data stored on the server.

### Step 19

Terminal displays cart summary and allows order confirmation.

Terminal requests cart summary data from the server and receives itemized cost and delivery information.

Input: cart summary data and associated metadata.

Terminal formats and displays the list of items, including quantities, prices, and total cost. Terminal provides a confirmation control that, when activated by the user, triggers either a redirect to the electronic commerce platform checkout page or a server-side checkout request, depending on configuration.

Output: user interface elements for confirming or modifying the prepared purchase.

### Step 20

User confirms the order and later provides feedback.

User completes the purchase by confirming on the terminal and, after the ingredients are delivered and meals are prepared, returns to the application to rate the dishes.

Input: recipe experience and satisfaction level.

User enters ratings and comments for each dish. Terminal encapsulates this evaluation information and sends it to the server. Server receives the evaluation information and appends it to feedback records. Server then incorporates the new feedback into subsequent training or fine-tuning of the preference model and adjusts feature representations of the user, which in turn influence future prompt sentences and selection logic.

Output: updated feedback data stored on the server and updated parameters for preference and health models that will improve future meal proposals.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

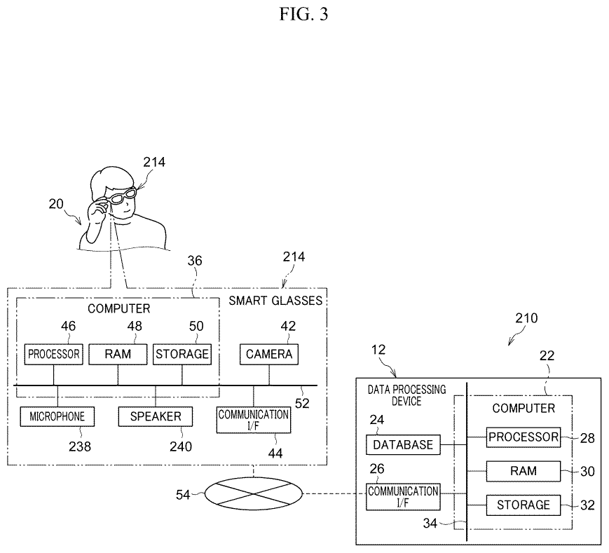

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

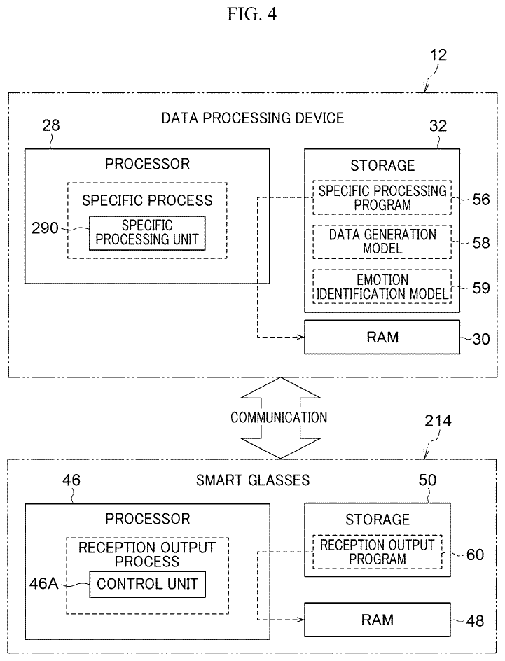

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

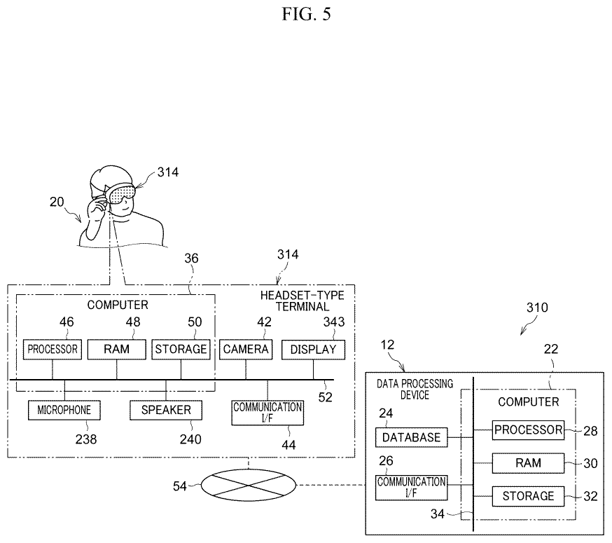

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

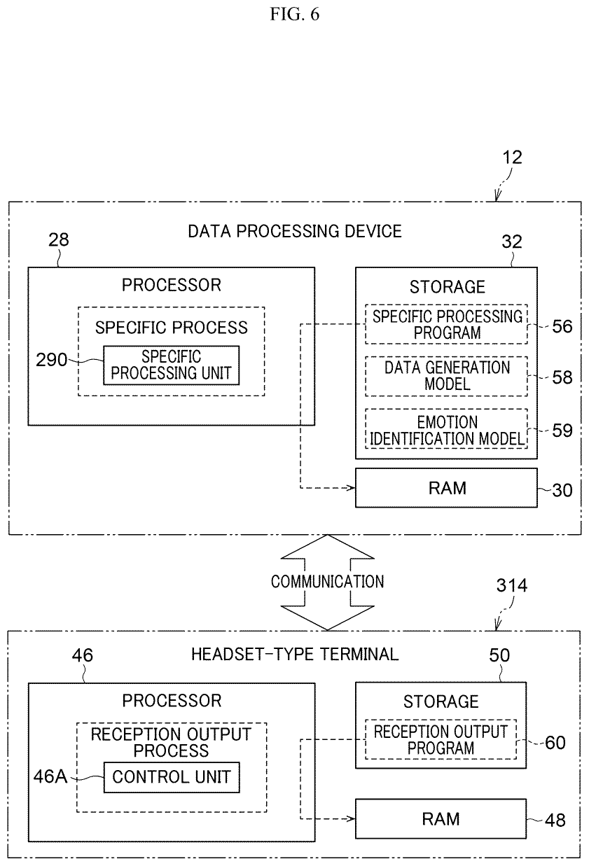

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

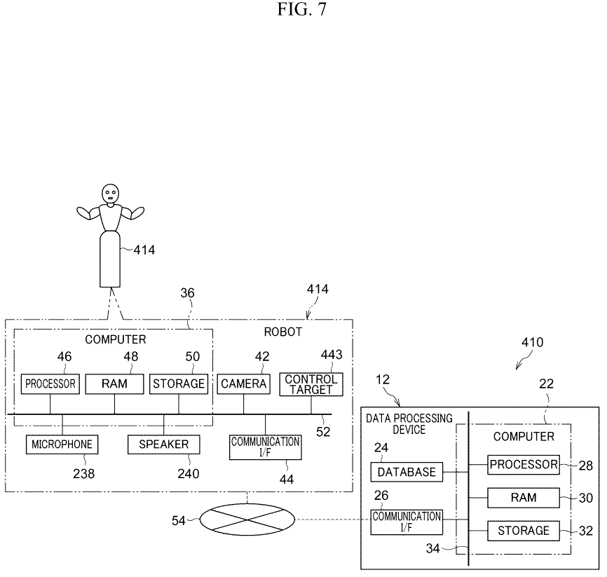

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

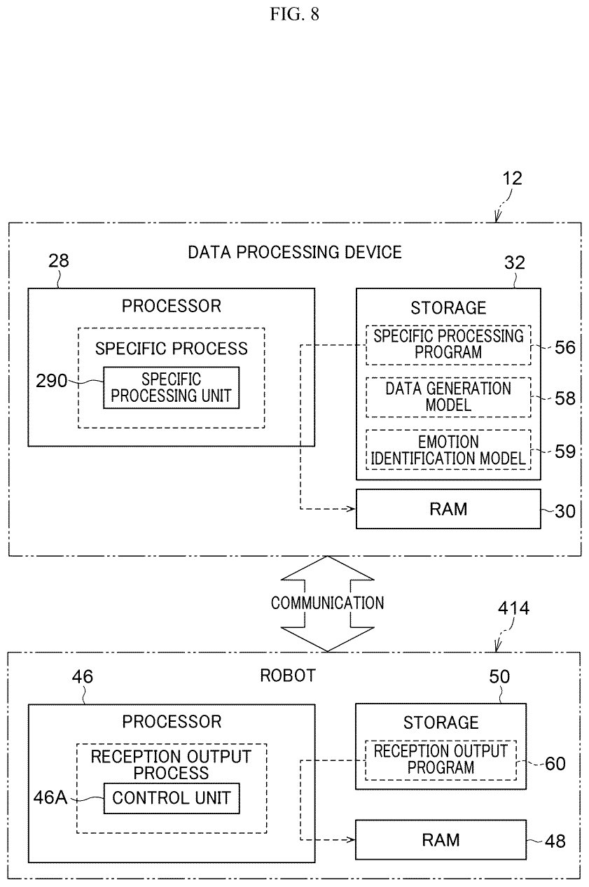

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

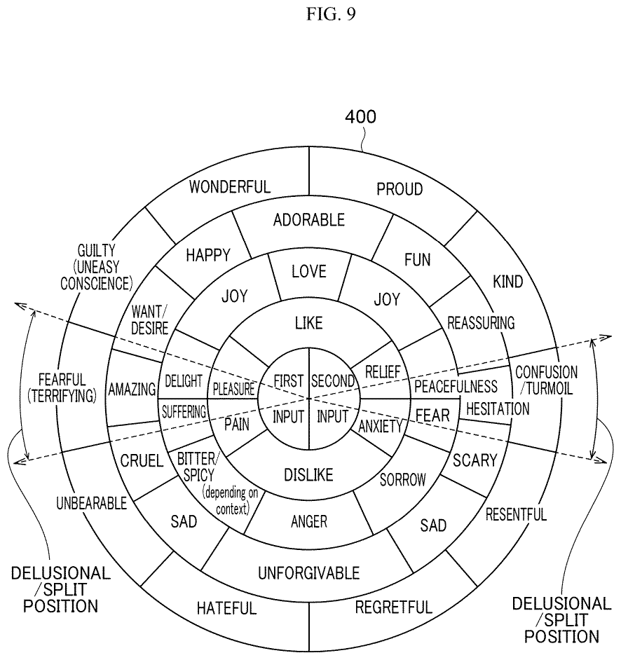

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

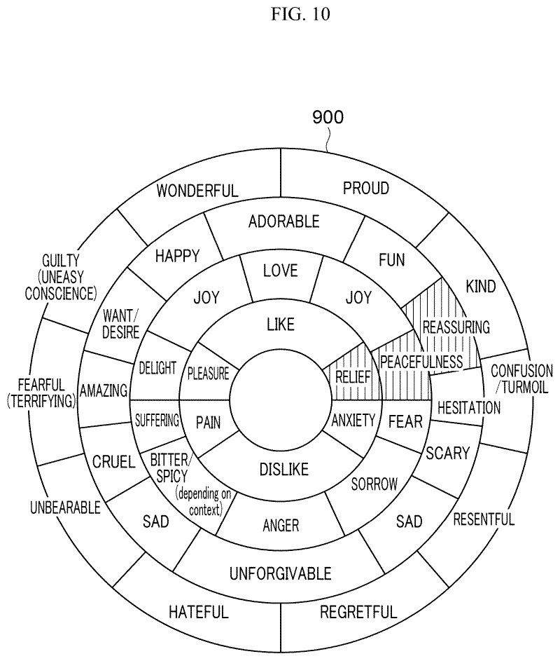

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
receive, via an input interface, budget information, preference information, and allergen information from a user,
generate, based on a condition including the budget information, the preference information, and the allergen information, a search request for searching meal information stored in an information storage device, acquire candidate meal information by executing the search request, and generate a prompt sentence including the candidate meal information and the condition, the prompt sentence being input to a generative AI model so as to cause the generative AI model to generate meal plan information for a predetermined period,
extract ingredient information for each meal included in the meal plan information, aggregate quantity information for each type of ingredient based on the ingredient information, and generate purchase candidate ingredient information,
transmit, based on the purchase candidate ingredient information, a product search request to an external product sales service through a communication network, acquire product information from the external product sales service, select purchase target product information for each ingredient from the product information, and add the purchase target product information to purchase management information of the external product sales service so as to generate purchase preparation information representing a state immediately before ordering, and
generate output information for presentation to a user device, the output information including the meal plan information and the purchase preparation information.

### Supplementary 2

The system according to supplementary 1,
wherein the processor is configured to
continuously input a learning prompt sentence including historical meal information and historical preference information of the user to the generative AI model, update an internal parameter or an operation condition of the generative AI model so as to cause the generative AI model to learn feature information indicating a meal preference and a health condition of the user, and optimize the meal plan information based on the feature information.

### Supplementary 3

The system according to supplementary 1,
wherein the processor is configured to
estimate an emotional state of the user based on operation information or sensor information of the user, and, in accordance with the emotional state, input to the generative AI model a prompt sentence including a condition relating to a configuration or a presentation format of the meal plan information so as to control output content of the generative AI model, or change at least one of a display order, an emphasis degree, and explanation content of the output information so as to optimize a presentation method of a recipe.

### Application Example 1

### Supplementary 1

A system comprising a processor,
wherein the processor is configured to
receive, from a user via a communication terminal, condition information including numerical information regarding a budget and attribute information regarding user preferences and restrictions,
analyze the condition information and, based on a result of the analysis, generate a prompt sentence that instructs generation of candidate dinner menus for a plurality of days and ingredient groups corresponding to the candidate dinner menus,
supply the prompt sentence as input to a generative AI model and execute inference processing by the generative AI model to obtain dinner menus for the plurality of days that satisfy the condition information and ingredient information included in the dinner menus,
aggregate the ingredient information included in the dinner menus for the plurality of days and generate, by performing data processing including normalization of ingredient names, conversion of units, and summation of quantities, ingredient-total information indicating total quantities per ingredient over the plurality of days,
generate, based on the ingredient-total information, order data including product identification information and order quantities corresponding to the ingredients by associating the ingredients with product information provided from an external commerce service, and transmit the order data to an application programming interface of the commerce service so as to prepare an order to a state immediately before placement, and
present the dinner menus for the plurality of days and the order data to the user via the communication terminal, receive confirmation and correction operations from the user, and reflect a result of the correction operations in the ingredient-total information and the order data.

### Supplementary 2

The system according to supplementary 1,
wherein the processor is configured to
use the generative AI model or another learning model to accumulate information including user meal history information, preference information, and health condition information, and, based on learning processing performed on the accumulated information, optimize the prompt sentence and contents of the dinner menus for the plurality of days for each user.

### Supplementary 3

The system according to supplementary 1,
wherein the processor is configured to
estimate an emotional state of the user based on input operation information, voice information, or image information acquired from the communication terminal, and, in accordance with the estimated emotional state, change at least one of a presentation order, a presentation expression, and proposal contents of the dinner menus for the plurality of days so as to dynamically control a presentation method of recipes.

### Example 2

### Supplementary 1

A system comprising a processor,
wherein the processor is configured to
receive, via a communication network from a user terminal, budget information, preference information, and allergen-avoidance information as input, and analyze the input information to extract meal-planning conditions; and
generate, based on the meal-planning conditions and nutritional-target information, a prompt sentence that instructs a generative AI model to generate cooking procedure information for meals for a plurality of days, transmit the prompt sentence to the generative AI model, and obtain the cooking procedure information for the plurality of days from the generative AI model; and
analyze the obtained cooking procedure information to extract, as structured data, meal information and ingredient information for each day, acquire nutrient data from a nutrient-information storage apparatus based on the ingredient information, and calculate nutrient amounts for each meal and for a predetermined period in total based on the structured data and the nutrient data; and
compare the calculated nutrient amounts with predetermined nutrient-intake criteria, automatically adjust the structured data such that a nutrient-intake balance falls within a target range by changing at least one of a kind and a quantity of ingredients included in the meal information based on a comparison result, and, when necessary, transmit an additional prompt sentence including an adjustment result to the generative AI model and obtain adjusted cooking procedure information; and
aggregate, based on the adjusted structured data, ingredients required for the predetermined period, generalize the ingredients as merchandise information, generate purchase-candidate information including the merchandise information and quantity information in accordance with an interface specification of an electronic transaction apparatus, and transmit the purchase-candidate information to the electronic transaction apparatus to generate a purchase-candidate state immediately before ordering; and
generate display data for transmitting, to the user terminal, the cooking procedure information, the nutrient amounts, and identification information related to the purchase-candidate state, and for enabling the user terminal to present the cooking procedure information as meal-planning information and to present the purchase-candidate information.

### Supplementary 2

The system according to supplementary 1,
wherein the processor is configured to cause the generative AI model, in generating the prompt sentence and in adjusting the structured data, to learn tendencies of a user's meal preferences and health conditions based on past input information and selection history of the user, and dynamically change contents of the prompt sentence so as to generate a meal plan for the predetermined period that simultaneously takes into account ingredient reusability, cost effectiveness, and nutrient-intake balance based on a learning result.

### Supplementary 3

The system according to supplementary 1,
wherein the processor is configured to estimate an emotional state based on operation-history information or biometric information acquired from the user terminal, and change at least one of a style and output condition of the prompt sentence to the generative AI model in accordance with the emotional state, thereby adjusting at least one of a presentation method of the meal-planning information and kinds of proposed meals.

### Application Example 2

### Supplementary 1

A system comprising a processor,
wherein the processor is configured to
acquire, as constraint information related to meals, budget information, ingredient-avoidance information, and allergen-avoidance information from a user through a terminal device,
acquire user information including the constraint information, health-related information including at least one of physical-activity information and nutritional-status information, and emotion-related information indicating an emotional state of the user,
analyze the user information and, based on a result of the analysis, generate a prompt sentence that instructs a generative AI model to generate a meal proposal for a predetermined period including a plurality of meal contents that satisfy the constraint information and the health-related information and that have characteristics corresponding to the emotion-related information,
input the prompt sentence to the generative AI model and obtain, from the generative AI model, a generation result including a plurality of cooking procedures constituting the meal proposal and ingredient information corresponding to the cooking procedures,
extract the ingredient information from the generation result, specify required articles based on the extracted ingredient information and item-identification information associated with merchandise-information records, and register the specified articles, through an electronic commerce platform, as purchase-processing information in a state immediately before order placement, and
generate presentation information for presenting the meal proposal to the terminal device based on the generation result and change a presentation mode of the presentation information in accordance with the emotion-related information.

### Supplementary 2

The system according to supplementary 1,
wherein the processor is configured to input evaluation information from the user regarding dishes included in the meal proposal and the health-related information as learning data to a machine-learning model, update parameters representing user preferences and health states by using the machine-learning model, and optimize at least one of the prompt sentence to be input to the generative AI model and a composition of dishes selected from the generation result based on the updated parameters.

### Supplementary 3

The system according to supplementary 1,
wherein the processor is configured to acquire emotion-estimation input information including acoustic information and image information, estimate the emotional state of the user by using at least one of speech-recognition processing and image-recognition processing, and change at least one of content of the prompt sentence, a presentation order of the meal proposal, and wording used in the presentation of the meal proposal in accordance with the estimated emotional state.

What is claimed is:

1. A system comprising:
circuitry configured to:
receive, via a communication interface coupled to a packet-switched network, constraint parameters including at least a numerical constraint parameter, a first exclusion parameter, and a second exclusion parameter from a terminal device, and generate a structured condition incorporating the constraint parameters stored in a storage device;
tokenize the structured condition, configure inference parameters including temperature and maximum output length, transmit the tokenized structured condition to an inference engine executing a generative AI model, and decode an output token sequence from the inference engine to obtain a structured output plan as a text string; and
parse the text string to extract record identifiers and attribute descriptions, normalize the extracted identifiers to canonical record identifiers, and transmit the structured output plan to the terminal device via the communication interface.

2. The system according to claim 1, wherein the circuitry is configured to receive the constraint parameters from the terminal device via the communication interface, parse the constraint parameters to extract the numerical constraint parameter, the first exclusion parameter, and the second exclusion parameter, and generate the structured condition incorporating the parsed parameters.

3. The system according to claim 2, wherein the circuitry is configured to generate a prompt sentence incorporating the structured condition and an instruction for the generative AI model to generate a structured output plan satisfying the numerical constraint parameter, the first exclusion parameter, and the second exclusion parameter, and transmit the prompt sentence to the inference engine.

4. The system according to claim 3, wherein the circuitry is configured to apply subword segmentation to the prompt sentence prior to tokenization, and encode the segmented prompt sentence using a vocabulary index of the generative AI model stored in the storage device.

5. The system according to claim 1, wherein the circuitry is configured to apply a scoring function to each record in the structured output plan by combining a constraint satisfaction score derived from the structured condition with a preference match score derived from user preference data stored in the storage device, and filter records having a combined score below a predetermined threshold.

6. The system according to claim 5, wherein the circuitry is configured to rank the records in the structured output plan by the combined score in descending order, and regenerate the prompt sentence and retransmit to the inference engine when a minimum number of qualifying records is not satisfied.

7. The system according to claim 1, wherein the circuitry is configured to receive biometric parameter data from the terminal device via the communication interface, incorporate the biometric parameter data into the structured condition, and generate a prompt sentence that instructs the generative AI model to generate a structured output plan satisfying biometric constraints in addition to the numerical constraint parameter and the first exclusion parameter.

8. The system according to claim 7, wherein the circuitry is configured to apply a machine learning model to the biometric parameter data to predict preference attributes, update the structured condition based on the predicted preference attributes, and incorporate the updated condition into the prompt sentence.

9. The system according to claim 1, wherein the circuitry is configured to perform multimodal estimation of an emotional state of the user from data received from the terminal device via the communication interface, and adjust the inference parameters and a presentation mode of the structured output plan based on the estimated emotional state.

10. The system according to claim 9, wherein the circuitry is configured to update the prompt sentence to modify at least one of a content selection criterion, a diversity setting, and a presentation format based on the estimated emotional state of the user.

11. The system according to claim 1, wherein the circuitry is configured to receive, from the terminal device, evaluation information indicating whether the structured output plan was accepted or modified by the user, incorporate the evaluation information into a feedback loop, and update a preference model stored in the storage device based on the evaluation information.

12. The system according to claim 11, wherein the circuitry is configured to feed updated parameters from the preference model back into the prompt sentence generation and inference parameters to pre-filter structured output plans in subsequent sessions.

13. The system according to claim 1, wherein the circuitry is configured to map the canonical record identifiers in the structured output plan to records available from an external data service via the communication interface, and generate fulfillment request data for transmission to the external data service.

14. The system according to claim 13, wherein the circuitry is configured to detect canonical record identifiers in the structured output plan that are absent from the external data service, generate supplementary fulfillment request data for the absent records, and transmit the supplementary fulfillment request data to an alternative data service via the communication interface.

15. The system according to claim 1, wherein the circuitry is configured to store the structured condition, the tokenized prompt sentence, and the structured output plan in the storage device with defined schemas, and use the stored data to regenerate or modify output plans without retransmitting to the inference engine.

16. The system according to claim 15, wherein the circuitry is configured to retrieve stored structured conditions and output plans from the storage device, and generate variant output plans by adjusting the numerical constraint parameter or the first exclusion parameter in the stored structured condition.

17. The system according to claim 1, wherein the circuitry is configured to monitor changes in the constraint parameters received from the terminal device over successive sessions, detect significant parameter shifts, and regenerate the prompt sentence for the inference engine when a detected shift exceeds a threshold.

18. A system comprising:
circuitry configured to:
receive, via a communication interface coupled to a packet-switched network, constraint parameters including a numerical constraint parameter, a first exclusion parameter, and a second exclusion parameter from a terminal device, and generate a structured condition incorporating the constraint parameters stored in a storage device;
tokenize the structured condition, configure inference parameters, transmit the tokenized structured condition to an inference engine executing a generative AI model, and decode an output token sequence to obtain a structured output plan as a text string;
parse the text string to extract record identifiers and attribute descriptions, normalize the extracted identifiers to canonical record identifiers, and apply a scoring function combining a constraint satisfaction score and a preference match score to rank the structured output plan; and
transmit the ranked structured output plan to the terminal device via the communication interface, and update a preference model stored in the storage device based on evaluation information received from the terminal device.

19. The system according to claim 18, wherein the circuitry is configured to map the canonical record identifiers in the ranked structured output plan to records available from an external data service via the communication interface, generate fulfillment request data, and transmit the fulfillment request data to the external data service.

20. A method comprising:
receiving, via a communication interface coupled to a packet-switched network, constraint parameters including at least a numerical constraint parameter, a first exclusion parameter, and a second exclusion parameter from a terminal device, and generating a structured condition incorporating the constraint parameters stored in a storage device;
tokenizing the structured condition, configuring inference parameters including temperature and maximum output length, transmitting the tokenized structured condition to an inference engine executing a generative AI model, and decoding an output token sequence from the inference engine to obtain a structured output plan as a text string; and
parsing the text string to extract record identifiers and attribute descriptions, normalizing the extracted identifiers to canonical record identifiers, and transmitting the structured output plan to the terminal device via the communication interface.
